(19) United States
(12) Patent Application Publication
(10) Pub. No.: US 2023/0025160 A1
(43) Pub. Date: Jan. 26, 2023

(54) CAR T CELLS WITH ENHANCED METABOLIC FITNESS

(71) Applicant: H. LEE MOFFITT CANCER CENTER AND RESEARCH INSTITUTE INC., Tampa, FL (US)

(72) Inventors: Frederick Locke, Lutz, FL (US); Reginald Atkins, Riverview, FL (US)

(21) Appl. No.: 17/756,386

(22) PCT Filed: Nov. 4, 2020
(86) PCT No.: PCT/US2020/058823

§ 371 Date: May 24, 2022

Related U.S. Application Data
(60) Provisional application No. 62/939,727, filed on Nov. 25, 2019.

(51) Int. Cl. C07K 14/47; C12N 5/0783

(52) U.S. Cl. CPC C07K 14/4702; C12N 5/0636; C07K 2319/33; C12N 2506/11; C12N 2510/00; A61K 2039/5156

(57) ABSTRACT

Disclosed herein are CAR-T cells engineered to express mutant PGC-1α, wildtype NT-PGC-1α, or mutant NT-PGC-1α to enhance or prevent degradation of metabolic fitness. Also disclosed herein is a method for enhancing metabolic fitness of a CAR-T cell by transducing the CAR-T cell with a vector encoding a mutant PGC-1α, wildtype NT-PGC-1α, or mutant NT-PGC-1α. Also disclosed is a method for producing CAR-T cells that involves transducing activated T cells with a viral vector encoding a mutant PGC-1α, wildtype NT-PGC-1α, or mutant NT-PGC-1α polypeptide.

### CROSS-REFERENCE TO RELATED APPLICATIONS

This application claims benefit of U.S. Provisional Application No. 62/939,727, filed Nov. 25, 2019, which is hereby incorporated herein by reference in its entirety.

### SEQUENCE LISTING

This application contains a sequence listing filed in electronic form as an ASCII.txt file entitled “320803-2460 Sequence Listing_ST25” created on Nov. 3, 2020. The content of the sequence listing is incorporated herein in its entirety.

### BACKGROUND

Insufficient persistence and effector function of chimeric antigen receptor (CAR) T cells have been challenging issues for adoptive T cell therapy. Generating potent CAR T cells is of increasing importance in the field.

### SUMMARY

Several factors are associated with CAR T efficacy. Proliferative capacity and asymmetric division of memory and naïve like phenotypes is required to supply enough cells to eradicate tumor cells and mediate disease remission. Greater persistence results in a reservoir of tumor specific T cells to surveil for disease over time increasing durability of response. Greater metabolic fitness allows CAR T cells to perform cytolytic and secretory function in a nutrient depleted microenvironment under high oxidative stress. The cells produce ATP via oxidative metabolism to slow or prevent differentiation toward terminally differentiated or exhausted effector phenoytpes. The cells also have reduced glycolytic flux. Persistent memory cells are characterized as having increased mitochondrial biomass with tubular morphology and a greater use of oxidative metabolism that relies more on the TCA cycle and ETC to produce ATP.

The percentage of central memory T cells or CCR7+CD45RO+ cells in peripheral blood after infusion also corelates with complete and durable responses. Central memory T cells are poised to proliferate and substantially contribute to peak expansion of CAR T cells after infusion and are therefore considered most desirable for therapy efficacy. Central memory T cells rely heavily on fatty acid oxidation and oxidative phosphorylation in mitochondria to synthesize ATP requiring augmented metabolic fitness. However, central memory T cells have a reduced capacity to lyse target cells relative to effector and effector memory CAR T cells. Modifying CAR T cells to increase their propensity to achieve central memory phenotypes is expected to contribute to improved therapy efficacy. However, there is a critical balance between metabolic fitness that must be targeted without sacrificing cytotoxic potential.

As disclosed herein, overexpressing PGC-1α, the master regulator of mitochondrial biogenesis and metabolic fitness, in CAR T cells significantly augments the frequency of central memory T cells when manufacturing CAR T cells. Importantly, these CAR T cells can possess the metabolic fitness required for expansion and survival in the tumor microenvironment without sacrificing cytotoxicity.

PGC-1α is a 798 a.a. transcriptional coactivator with no DNA binding domain or enzymatic activity. As a coactivator, it can bind with a broad set of transcription factors and induce the upregulation of many complex transcriptional programs. These transcriptional programs combine to initiate mitochondrial biogenesis, enhance oxidative metabolism (Fatty Acid Oxidation, TCA cycle, and Electron Transport Chain (ETC), mitochondrial flux (mitophagy), and reduce oxidative stress.

A multitude of post translational modifications (PTM) determine PGC-1α localization, activity, and rate of proteasomal degradation. At rest, constitutively expressed GSK3β ubiquitinates PGC-1α for proteasomal degradation as well as ETC complex subunits within the mitochondria, during which CAR T cells are vulnerable to a reduction in PGC-1α mediated metabolic fitness. During CAR and TCR signaling the PI3K signaling axis activates Akt, which phosphorylates and deactivates GSK3β and deactivates PGC-1α.

As disclosed herein, selectively targeting PGC-1α PTMs enhances or prevents degradation of metabolic fitness. Targeted PTMs include: Akt phosphorylation of PGC-1α at S571 suppresses PGC-1α activity; GSK3β—ubiquitinates PGC-1α (T295) for proteosomal degradation; Clk-2 phosphorylation of PGC-1α at S569, S571, S573, S577, S579, S581, S599, S616, S624, S629, S636 suppresses PGC-1α activity; and S6K phosphorylation of PGC-1α at S569 suppresses PGC-1α activity.

Therefore, disclosed herein are CAR-T cells engineered to express a mutant PGC-1α, a wildtype NT-PGC-1α, or a mutant NT-PGC-1α polypeptide to enhance or prevent degradation of metabolic fitness.

Also disclosed herein is a vector comprising nucleic acid sequences encoding both a CAR polypeptide and a mutant PGC-1α, a wildtype NT-PGC-1α, or a mutant NT-PGC-1α polypeptide. In some embodiments, the CAR polypeptide and mutant PGC-1α, a wildtype NT-PGC-1α, or a mutant NT-PGC-1α polypeptide are operably linked to the same promoter. For example, the nucleic acids encoding the CAR polypeptide and the mutant PGC-1α, a wildtype NT-PGC-1α, or a mutant NT-PGC-1α polypeptide can be separated by a cleavable linker or IRES sequence.

In some embodiments, the mutant PGC-1α has an amino acid mutation at T295, S571, S569, S573, S577, S579, S581, S599, S616, S624, S629, S636, or any combination thereof. In some embodiments, the mutant PGC-1α has the amino acid sequence SEQ ID NO:3, SEQ ID NO:4, or SEQ ID NO:5. In some embodiments, the wildtype NT-PGC-1α has the amino acid sequence SEQ ID NO:2. In some embodiments, the mutant NT-PGC-1α has an amino acid mutation at L29, L33, L36, L38, K78, L92, L96, L99, V101, K145, V183, K184, T185, E186, S195, S242, K254, T257, T263, S266, L269, or any combination thereof. In some embodiments, the mutant PGC-1α has the amino acid sequence SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, or SEQ ID NO:15.

Also disclosed herein is a method for enhancing metabolic fitness of a CAR-T cell, comprising transducing the CAR-T cell with a vector encoding a mutant PGC-1α, a NT-PGC-1α, or mutant NT-PGC-1α disclosed herein.

Also disclosed is a method for producing CAR-T cells, that involves isolating PBMCs from a donor, isolating T cells from the PBMCs, stimulating the T cells with CD3/CD28 beads, transducing the activated T cells with a viral vector encoding a CAR polypeptide, transducing the activated T cells with a viral vector encoding a mutant PGC-1α, a NT-PGC-1α, or mutant NT-PGC-1α polypeptide disclosed herein, and expanding the CAR-T cells.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A and 1B show baseline (BL) and post-co-retroviral transduction percentages of central memory CAR T cells (Tcm) (FIG. 1A) and effector memory CAR T cells (Tem) (FIG. 1B). Tcm and Tem phenotypes were quantified via flow cytometry after 7-10 day transduction and isolation of CAR+and mutant PGC-1α+T cells using flow cytometry based cell sorting. Central memory CAR T cells were defined as double positive for CCR7 (CD197) and CD45RO surface antigens. Effector memory CAR T cells were defined as negative for CCR7 and positive for CD45RO surface antigens. Transduction with mutant PGC-1α or empty vector (EV) was verified with DsRed Express fluorescent protein reporter gene expression. CAR transduction was verified with truncated (non-functional) CD34 surface receptor reporter gene expression. Empty vector+DsRed Express fluorescent protein (EV), PGC-1α S570A (A mutant—resistant to deactivating phosphorylation by Akt), PGC-1α T295A (G mutant—resistant to ubiquitination by GSK3β), PGC-1α S570A, T295A (GA mutant—resistant to deactivating phosphorylation by Akt and resistant to ubiquitination by GSK3β and subsequent proteasomal degradation).

FIG. 2 shows baseline (BL) and post-co-transduction percentages of effector CAR T cells (Teff) cells were isolated and analyzed as previously mentioned. Teff pheonytpe percentages were verified with flow cytometry as being double negative for CCR7 and CD45RO surface antigens.

FIG. 3 shows relative amounts of PGC-1α assessed by flow cytometry for EV, G, A, and GA mutants 48 hours after in vitro stimulation with immobilized CD-19 protein.

FIG. 4 shows relative amounts of mitochondrial resident superoxide dismutase 2 (SOD-2) assessed by flow cytometry for EV, G, A, and GA mutants 48 hours after in vitro stimulation with immobilized CD-19 protein.

FIG. 5 shows relative amounts of mitofusin 2 (Mfn2) assessed by flow cytometry for EV, G, A, and GA mutants 48 hours after in vitro stimulation with immobilized CD-19 protein.

FIG. 6 shows relative amounts of nuclear respiratory factor 2 (NRF-2) assessed by flow cytometry for EV, G, A, and GA mutants 48 hours after in vitro stimulation with immobilized CD-19 protein.

FIG. 7 shows relative amounts of PGC-1α assessed by flow cytometry for EV, G, A, and GA mutants 48 hours after in vitro stimulation with CD-19 expressing K562 cell line at a ratio of 2 CAR T to 1 Target cell. Unstimulated (US).

FIG. 8 shows relative amounts of c-FLIP assessed by flow cytometry for EV, G, A, and GA mutants 48 hours after in vitro stimulation with CD-19 expressing K562 cell line at a ratio of 2 CAR T to 1 Target cell.

FIG. 9 shows relative amounts of cleaved caspase 3/7 assessed by flow cytometry for EV, G, A, and GA mutants 48 hours after in vitro stimulation with CD-19 expressing K562 cell line at a ratio of 2 CART to 1 Target cell.

FIGS. 10A and 10B show FACS analysis of CAR T cell subset distribution after manufacture of 28z CAR T cells with or without co-expression of PGC-1α variants.

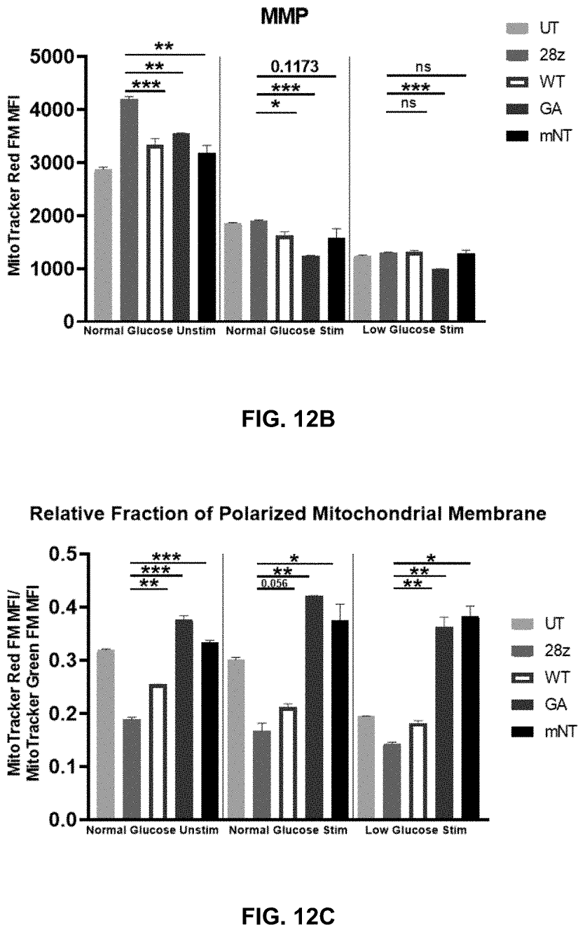

FIGS. 11A to 11C show FACS analysis quantification of PGC-1α target gene expression at rest and after stimulation with CD19 target baring cells. Estrogen Related Receptor α (ERRα), Nuclear Respiratory Factor 2 (NRF2), and Mitochondrial Transcription Factor A (TFAM) are transcription factors upregulated by PGC-1α that drive the transcription of many genes needed for mitochondrial biogenesis (making more mitochondria), mitophagy (mitochondrial quality control), and metabolic fitness—the ability to synthesize ATP in different ways (e.g. glycolysis vs oxidative phosphorylation). The point of overexpressing PGC-1α is it targets a vast array of transcription factors that amplify its impact on energy homeostasis and mitochondrial biogenesis. The expression of these representative PGC-1α is statistically increased in all PGC-1α CAR T but increased most in NT and mNT expressing variants.

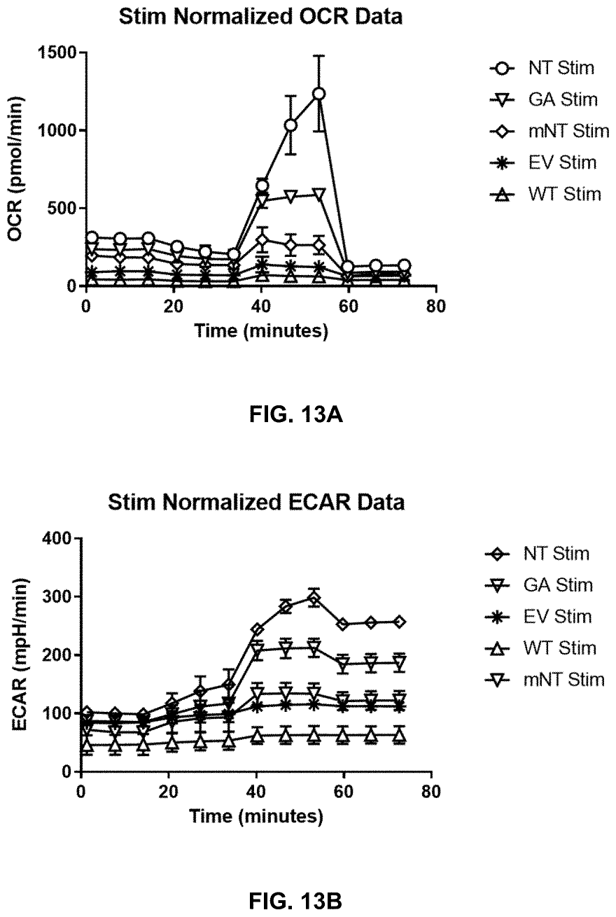

FIGS. 12A to 12C show relative total mitochondrial biomass (FIG. 12A), polarized (functional) mitochondrial biomass (FIG. 12B), and the ratio of functional mitochondrial biomass to total biomass (FIG. 12C) of CAR T cells with or without PGC-1α overexpression. Although PGC-1α is usually mentioned in the context of mitochondrial biogenesis, an often overlooked role is in enhancing mitochondrial quality control which is critical for the removal of damaged or non-functioning mitochondria.

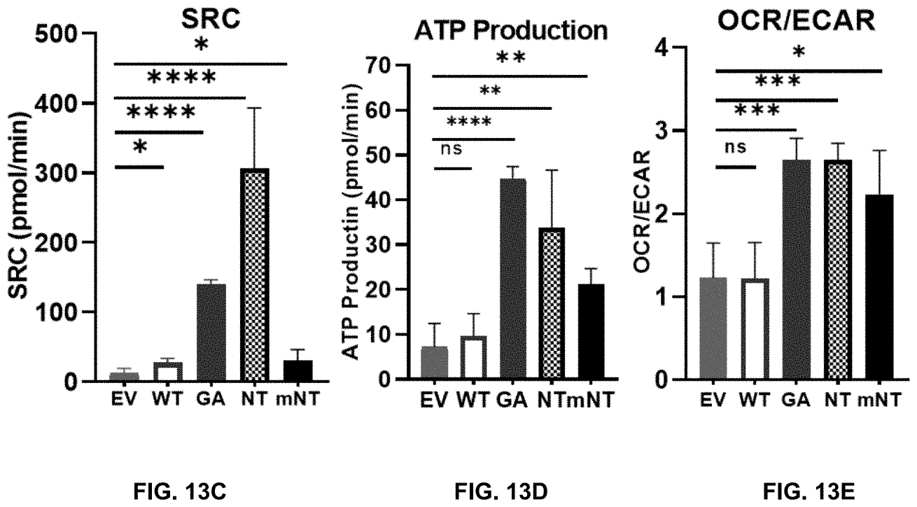

FIGS. 13A to 13E show mitochondrial stress test performed using a Seahorse Flux Analyzer to quantify Oxygen consumption rate (OCR) (FIG. 13A), Extracellular Acidification Rate (ECAR) (FIG. 13B), Spare Respiratory Capacity (SRC) (FIG. 13C), OCR/ECAR ratio (FIG. 13D), and mitochondrial ATP production (FIG. 13E) for Empty Vector CAR T cells and PGC-1α overexpressing CAR T cells. SRC represents the ability for a cell to synthesize and meet energy demands under stress which is critical for T cell cytotoxicity and proliferation in the tumor microenvironment (tme). An increased OCR/ECAR ratio indicates enhanced metabolic flexibility and a reduced reliance on glucose, a fuel source that is scarce in the tme. Increased ATP production from mitochondria represents an overall increase in basal ATP synthesis from mitochondria again critically essential for CAR T cell function and survival.

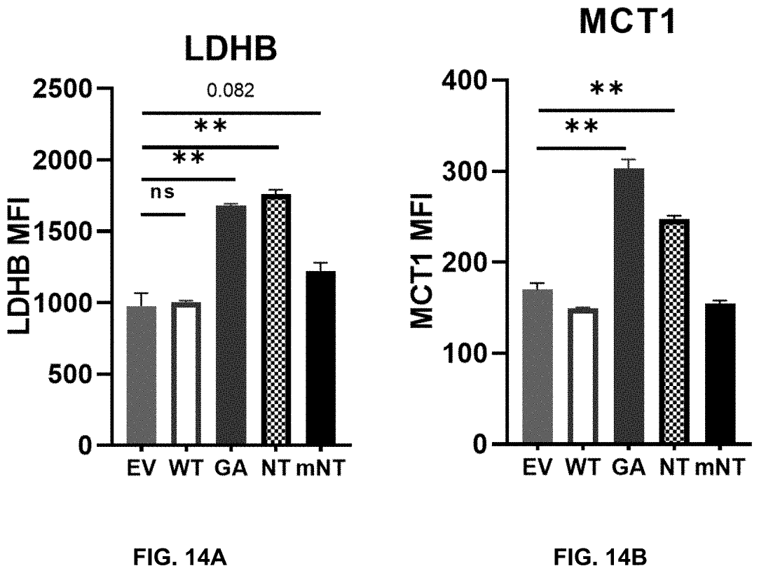

FIGS. 14A and 14B show Lactate Dehydrogenase B (LDHB) (FIG. 14A) and Monocarboxylate Transporter 1 (MCT1) (FIG. 14B) protein levels are increased in PGC-1α overexpressing CAR T relative to control EV CAR T. Lactate is a suppressive metabolite that also contributes to acidosis in the tme. CAR T cells with upregulated lactate transporters and LDHB, which converts lactate into pyruvate (which can be used to synthesize ATP), equips CAR T with the capacity to use a toxic metabolite for ATP synthesis. Removal of lactate from the tme could also restore pH and reinvigorate other immune cells in the tme contributing to enhanced tumor immunity.

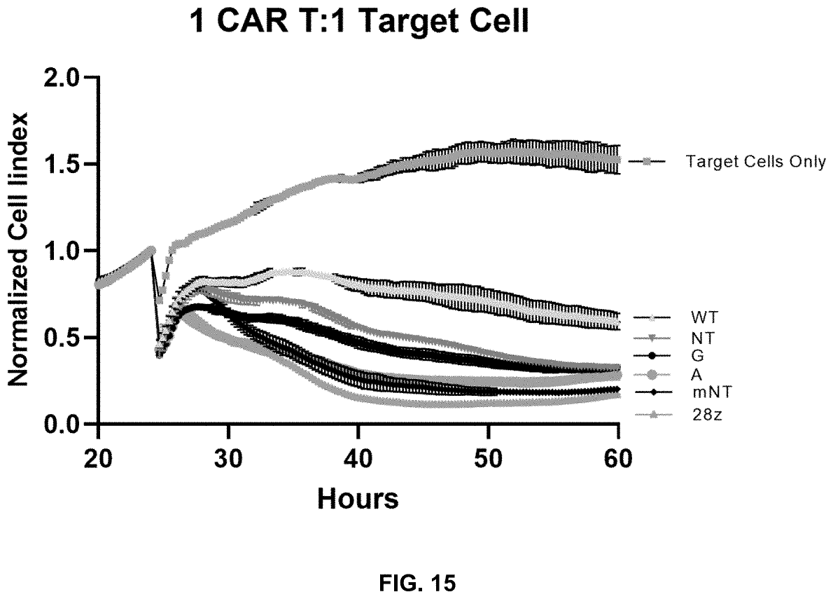

FIG. 15 shows real time cytotoxicity of CAR T cells with or without PGC-1α overexpression using a xCelligence RTCA device. CAR T cells were co-cultured at 1 to 1 CAR T cell to Target ratio. Overexpression of PGC-1α did not significantly attenuate CAR T cytotoxicity in A, G, NT, or mNT variants. Cytotoxicity of mNT was equivalent to 28z (control CAR T).

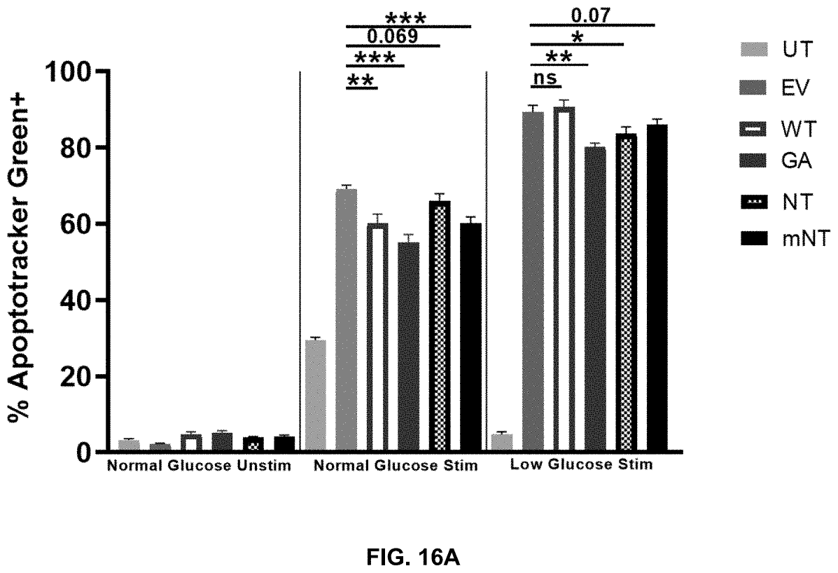

FIG. 16A shows the percentage of apoptotic CAR T cells before activation in normal glucose media and after activat with CD19 expressing K562 target cells in normal and low glucose (0.1 mM) media. FIG. 16B shows total oxidative stress measured with CelIROX in CAR T cells before activation in normal glucose media (10 mM) and after activation with CD19 expressing K562 target cells in normal and low glucose (0.1 mM) media. Wild type and mutant PGC-1α overexpression upregulates antioxidant enzymes providing CAR T cells with a survival advantage by neutralizing destructive reactive oxygen species (ROS).

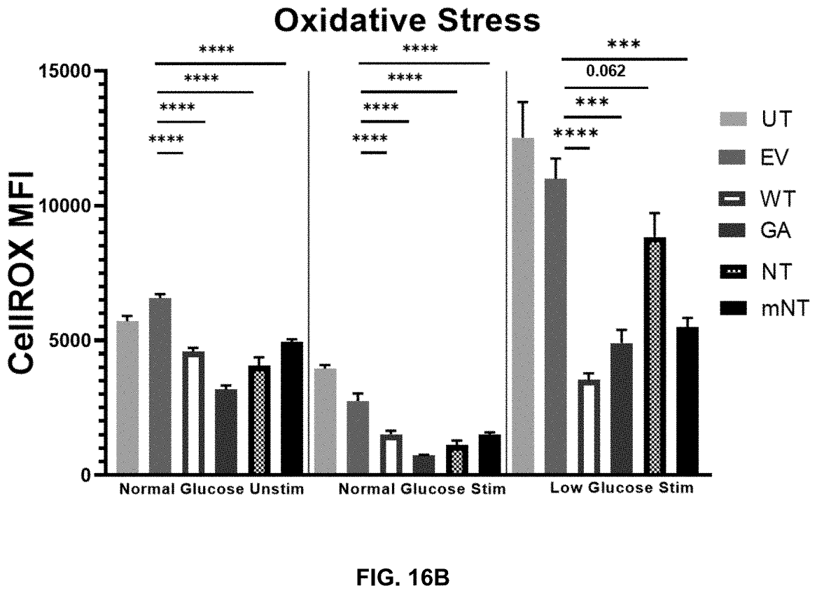

FIGS. 17A and 17B show the percentage of CCR7+ (FIG. 17A) and PD-1+ (FIG. 17B) CAR T cells after 48 hours of stimulation with immobilized CD19 protein. FIG. 17C shows fold expansion of Control (EV) and PGC-1α overexpressing CAR T cells after 48 hours of stimulation with immobilized CD19 protein. CCR7 is lymphatic tissue homing surface protein that aids in CAR T localization to lymphnodes and lymphatic tissue where tumor often resides. Programmed death receptor 1 (PD-1) an exhaustion marker that is a surrogate for T cell dysfunction and hyporesponsiveness. Less exhausted CAR T cells function better and provide improved anti-tumor activity. Capacity of CAR T cells to expand after activation is critical for clinical efficacy and is positively correlated with complete response and durability of responses.

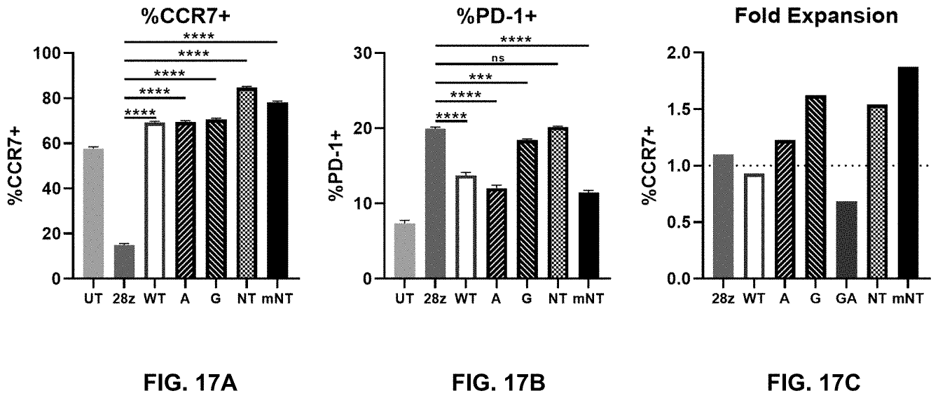

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

The term “amino acid sequence” refers to a list of abbreviations, letters, characters or words representing amino acid residues. The amino acid abbreviations used herein are conventional one letter codes for the amino acids and are expressed as follows: A, alanine; B, asparagine or aspartic acid; C, cysteine; D aspartic acid; E, glutamate, glutamic acid; F, phenylalanine; G, glycine; H histidine; I isoleucine; K, lysine; L, leucine; M, methionine; N, asparagine; P, proline; Q, glutamine; R, arginine; S, serine; T, threonine; V, valine; W, tryptophan; Y, tyrosine; Z, glutamine or glutamic acid.

The term “antibody” refers to an immunoglobulin, derivatives thereof which maintain specific binding ability, and proteins having a binding domain which is homologous or largely homologous to an immunoglobulin binding domain. These proteins may be derived from natural sources, or partly or wholly synthetically produced. An antibody may be monoclonal or polyclonal. The antibody may be a member of any immunoglobulin class from any species, including any of the human classes: IgG, IgM, IgA, IgD, and IgE. In exemplary embodiments, antibodies used with the methods and compositions described herein are derivatives of the IgG class. In addition to intact immunoglobulin molecules, also included in the term “antibodies” are fragments or polymers of those immunoglobulin molecules, and human or humanized versions of immunoglobulin molecules that selectively bind the target antigen.

The term “antibody fragment” refers to any derivative of an antibody which is less than full-length. In exemplary embodiments, the antibody fragment retains at least a significant portion of the full-length antibody's specific binding ability. Examples of antibody fragments include, but are not limited to, Fab, Fab′, F(ab′)2, scFv, Fv, dsFv diabody, Fc, and Fd fragments. The antibody fragment may be produced by any means. For instance, the antibody fragment may be enzymatically or chemically produced by fragmentation of an intact antibody, it may be recombinantly produced from a gene encoding the partial antibody sequence, or it may be wholly or partially synthetically produced. The antibody fragment may optionally be a single chain antibody fragment. Alternatively, the fragment may comprise multiple chains which are linked together, for instance, by disulfide linkages. The fragment may also optionally be a multimolecular complex. A functional antibody fragment will typically comprise at least about 50 amino acids and more typically will comprise at least about 200 amino acids.

The term “antigen binding site” refers to a region of an antibody that specifically binds an epitope on an antigen.

The term “aptamer” refers to oligonucleic acid or peptide molecules that bind to a specific target molecule. These molecules are generally selected from a random sequence pool. The selected aptamers are capable of adapting unique tertiary structures and recognizing target molecules with high affinity and specificity. A “nucleic acid aptamer” is a DNA or RNA oligonucleic acid that binds to a target molecule via its conformation, and thereby inhibits or suppresses functions of such molecule. A nucleic acid aptamer may be constituted by DNA, RNA, or a combination thereof. A “peptide aptamer” is a combinatorial protein molecule with a variable peptide sequence inserted within a constant scaffold protein. Identification of peptide aptamers is typically performed under stringent yeast dihybrid conditions, which enhances the probability for the selected peptide aptamers to be stably expressed and correctly folded in an intracellular context.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

The term “chimeric molecule” refers to a single molecule created by joining two or more molecules that exist separately in their native state. The single, chimeric molecule has the desired functionality of all of its constituent molecules. One type of chimeric molecules is a fusion protein.

The term “engineered antibody” refers to a recombinant molecule that comprises at least an antibody fragment comprising an antigen binding site derived from the variable domain of the heavy chain and/or light chain of an antibody and may optionally comprise the entire or part of the variable and/or constant domains of an antibody from any of the Ig classes (for example IgA, IgD, IgE, IgG, IgM and IgY).

The term “epitope” refers to the region of an antigen to which an antibody binds preferentially and specifically. A monoclonal antibody binds preferentially to a single specific epitope of a molecule that can be molecularly defined. In the present invention, multiple epitopes can be recognized by a multispecific antibody.

The term “fusion protein” refers to a polypeptide formed by the joining of two or more polypeptides through a peptide bond formed between the amino terminus of one polypeptide and the carboxyl terminus of another polypeptide. The fusion protein can be formed by the chemical coupling of the constituent polypeptides or it can be expressed as a single polypeptide from nucleic acid sequence encoding the single contiguous fusion protein. A single chain fusion protein is a fusion protein having a single contiguous polypeptide backbone. Fusion proteins can be prepared using conventional techniques in molecular biology to join the two genes in frame into a single nucleic acid, and then expressing the nucleic acid in an appropriate host cell under conditions in which the fusion protein is produced.

The term “Fab fragment” refers to a fragment of an antibody comprising an antigen-binding site generated by cleavage of the antibody with the enzyme papain, which cuts at the hinge region N-terminally to the inter-H-chain disulfide bond and generates two Fab fragments from one antibody molecule.

The term “F(ab′)2 fragment” refers to a fragment of an antibody containing two antigen-binding sites, generated by cleavage of the antibody molecule with the enzyme pepsin which cuts at the hinge region C-terminally to the inter-H-chain disulfide bond.

The term “Fc fragment” refers to the fragment of an antibody comprising the constant domain of its heavy chain.

The term “Fv fragment” refers to the fragment of an antibody comprising the variable domains of its heavy chain and light chain.

“Gene construct” refers to a nucleic acid, such as a vector, plasmid, viral genome or the like which includes a “coding sequence” for a polypeptide or which is otherwise transcribable to a biologically active RNA (e.g., antisense, decoy, ribozyme, etc), may be transfected into cells, e.g. in certain embodiments mammalian cells, and may cause expression of the coding sequence in cells transfected with the construct. The gene construct may include one or more regulatory elements operably linked to the coding sequence, as well as intronic sequences, polyadenylation sites, origins of replication, marker genes, etc.

The term “identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting. For example, polypeptides having at least 70%, 85%, 90%, 95%, 98% or 99% identity to specific polypeptides described herein and preferably exhibiting substantially the same functions, as well as polynucleotide encoding such polypeptides, are contemplated. Unless otherwise indicated a similarity score will be based on use of BLOSUM62. When BLASTP is used, the percent similarity is based on the BLASTP positives score and the percent sequence identity is based on the BLASTP identities score. BLASTP “Identities” shows the number and fraction of total residues in the high scoring sequence pairs which are identical; and BLASTP “Positives” shows the number and fraction of residues for which the alignment scores have positive values and which are similar to each other. Amino acid sequences having these degrees of identity or similarity or any intermediate degree of identity of similarity to the amino acid sequences disclosed herein are contemplated and encompassed by this disclosure. The polynucleotide sequences of similar polypeptides are deduced using the genetic code and may be obtained by conventional means, in particular by reverse translating its amino acid sequence using the genetic code.

The term “linker” is art-recognized and refers to a molecule or group of molecules connecting two compounds, such as two polypeptides. The linker may be comprised of a single linking molecule or may comprise a linking molecule and a spacer molecule, intended to separate the linking molecule and a compound by a specific distance.

The term “multivalent antibody” refers to an antibody or engineered antibody comprising more than one antigen recognition site. For example, a “bivalent” antibody has two antigen recognition sites, whereas a “tetravalent” antibody has four antigen recognition sites. The terms “monospecific”, “bispecific”, “trispecific”, “tetraspecific”, etc. refer to the number of different antigen recognition site specificities (as opposed to the number of antigen recognition sites) present in a multivalent antibody. For example, a “monospecific” antibody's antigen recognition sites all bind the same epitope. A “bispecific” antibody has at least one antigen recognition site that binds a first epitope and at least one antigen recognition site that binds a second epitope that is different from the first epitope. A “multivalent monospecific” antibody has multiple antigen recognition sites that all bind the same epitope. A “multivalent bispecific” antibody has multiple antigen recognition sites, some number of which bind a first epitope and some number of which bind a second epitope that is different from the first epitope.

The term “nucleic acid” refers to a natural or synthetic molecule comprising a single nucleotide or two or more nucleotides linked by a phosphate group at the 3′ position of one nucleotide to the 5′ end of another nucleotide. The nucleic acid is not limited by length, and thus the nucleic acid can include deoxyribonucleic acid (DNA) or ribonucleic acid (RNA).

The term “operably linked to” refers to the functional relationship of a nucleic acid with another nucleic acid sequence. Promoters, enhancers, transcriptional and translational stop sites, and other signal sequences are examples of nucleic acid sequences operably linked to other sequences. For example, operable linkage of DNA to a transcriptional control element refers to the physical and functional relationship between the DNA and promoter such that the transcription of such DNA is initiated from the promoter by an RNA polymerase that specifically recognizes, binds to and transcribes the DNA.

The terms “peptide,” “protein,” and “polypeptide” are used interchangeably to refer to a natural or synthetic molecule comprising two or more amino acids linked by the carboxyl group of one amino acid to the alpha amino group of another.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The terms “polypeptide fragment” or “fragment”, when used in reference to a particular polypeptide, refers to a polypeptide in which amino acid residues are deleted as compared to the reference polypeptide itself, but where the remaining amino acid sequence is usually identical to that of the reference polypeptide. Such deletions may occur at the amino-terminus or carboxy-terminus of the reference polypeptide, or alternatively both. Fragments typically are at least about 5, 6, 8 or 10 amino acids long, at least about 14 amino acids long, at least about 20, 30, 40 or 50 amino acids long, at least about 75 amino acids long, or at least about 100, 150, 200, 300, 500 or more amino acids long. A fragment can retain one or more of the biological activities of the reference polypeptide. In various embodiments, a fragment may comprise an enzymatic activity and/or an interaction site of the reference polypeptide. In another embodiment, a fragment may have immunogenic properties.

The term “protein domain” refers to a portion of a protein, portions of a protein, or an entire protein showing structural integrity; this determination may be based on amino acid composition of a portion of a protein, portions of a protein, or the entire protein.

The term “single chain variable fragment or scFv” refers to an Fv fragment in which the heavy chain domain and the light chain domain are linked. One or more scFv fragments may be linked to other antibody fragments (such as the constant domain of a heavy chain or a light chain) to form antibody constructs having one or more antigen recognition sites.

A “spacer” as used herein refers to a peptide that joins the proteins comprising a fusion protein. Generally a spacer has no specific biological activity other than to join the proteins or to preserve some minimum distance or other spatial relationship between them. However, the constituent amino acids of a spacer may be selected to influence some property of the molecule such as the folding, net charge, or hydrophobicity of the molecule.

The term “specifically binds”, as used herein, when referring to a polypeptide (including antibodies) or receptor, refers to a binding reaction which is determinative of the presence of the protein or polypeptide or receptor in a heterogeneous population of proteins and other biologics. Thus, under designated conditions (e.g. immunoassay conditions in the case of an antibody), a specified ligand or antibody “specifically binds” to its particular “target” (e.g. an antibody specifically binds to an endothelial antigen) when it does not bind in a significant amount to other proteins present in the sample or to other proteins to which the ligand or antibody may come in contact in an organism. Generally, a first molecule that “specifically binds” a second molecule has an affinity constant (Ka) greater than about 10⁵ M⁻¹ (e.g., 10⁶ M⁻¹, 10⁷ M⁻¹, 10⁸ M⁻¹, 10⁹ M⁻¹, 10¹⁰ M⁻¹, 10¹¹ M⁻¹, and 10¹² M⁻¹ or more) with that second molecule.

The term “specifically deliver” as used herein refers to the preferential association of a molecule with a cell or tissue bearing a particular target molecule or marker and not to cells or tissues lacking that target molecule. It is, of course, recognized that a certain degree of non-specific interaction may occur between a molecule and a non- target cell or tissue. Nevertheless, specific delivery, may be distinguished as mediated through specific recognition of the target molecule. Typically specific delivery results in a much stronger association between the delivered molecule and cells bearing the target molecule than between the delivered molecule and cells lacking the target molecule.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The terms “transformation” and “transfection” mean the introduction of a nucleic acid, e.g., an expression vector, into a recipient cell including introduction of a nucleic acid to the chromosomal DNA of said cell.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “variant” refers to an amino acid or peptide sequence having conservative amino acid substitutions, non-conservative amino acid substitutions (i.e. a degenerate variant), substitutions within the wobble position of each codon (i.e. DNA and RNA) encoding an amino acid, amino acids added to the C-terminus of a peptide, or a peptide having 60%, 70%, 80%, 90%, 95%, 96%, 97%, 98%, 99% sequence identity to a reference sequence.

The term “vector” refers to a nucleic acid sequence capable of transporting into a cell another nucleic acid to which the vector sequence has been linked. The term “expression vector” includes any vector, (e.g., a plasmid, cosmid or phage chromosome) containing a gene construct in a form suitable for expression by a cell (e.g., linked to a transcriptional control element).

### Mutant PGC-1α Proteins

As disclosed herein, selectively targeting PGC-1α PTMs enhances or prevents degradation of metabolic fitness. Therefore, disclosed herein are CAR-T cells engineered to express mutant PGC-1α to enhance or prevent degradation of metabolic fitness.

In some embodiments, the human PGC-1α has the amino acid sequence:

| (SEQ ID NO: 1) | | | | |
|---|---|---|---|---|
| | MAWDMCNQDS | ESVWSDIECA | ALVGEDQPLC | PDLPELDLSE |
| | LDVNDLDTDS | FLGGLKWCSD | QSEIISNQYN | NEPSNIFEKI |
| | DEENEANLLA | VLTETLDSLP | VDEDGLPSED | ALTDGDVTTD |
| | NEASPSSMPD | GTPPPQEAEE | PSLLKKLLLA | PANTQLSYNE |
| | CSGLSTQNHA | NHNHRIRTNP | AIVKTENSWS | NKAKSICQ00 |
| | KPQRRPCSEL | LKYLTTNDDP | PHTKPTENRN | SSRDKCTSKK |
| | KSHTQSQSQH | LQAKPTTLSL | PLTPESPNDP | KGSPFENKTI |
| | ERTLSVELSG | TAGLTPPTTP | PHKANQDNPF | RASPKLKSSC |
| | KTVVPPPSKK | PRYSESSGTQ | GNNSTKKGPE | QSELYAQLSK |
| | SSVLTGGHEE | RKTKRPSLRL | FGDHDYCQSI | NSKTEILINI |
| | SQELQDSRQL | ENKDVSSDWQ | GQICSSTDSD | QCYLRETLEA |
| | SKQVSPCSTR | KQLQDQEIRA | ELNKHFGHPS | QAVFDDEADK |
| | TGELRDSDFS | NEQFSKLPMF | INSGLAMDGL | FDDSEDESDK |
| | LSYPWDGTQS | YSLFNVSPSC | SSFNSPCRDS | VSPPKSLFSQ |
| | RPQRMRSRSR | SFSRHRSCSR | SPYSRSRSRS | PGSRSSSRSC |
| | YYYESSHYRH | RTHRNSPLYV | RSRSRSPYSR | RPRYDSYEEY |
| | QHERLKREEY | RREYEKRESE | RAKQRERQRQ | KAIEERRVIY |
| | VGKIRPDTTR | TELRDRFEVF | GEIEECTVNL | RDDGDSYGFI |
| | TYRYTCDAFA | ALENGYTLRR | SNETDFELYF | CGRKQFFKSN |
| | YADLDSNSDD | FDPASTKSKY | DSLDFDSLLK | EAQRSLRR. |

In some embodiments, there is a mutation in a GSK3β-dependent ubiquitination site. Ubiquitination inhibits activity by promoting proteosomal degradation. Therefore, in some embodiments, there is a mutation to residue T295 of SEQ ID NO:1. Therefore, in some embodiments, the mutant PGC-1α comprises the amino acid sequence:

| | | | |
|---|---|---|---|
| MAWDMCNQDS | ESVWSDIECA | ALVGEDQPLC | PDLPELDLSE |
| LDVNDLDTDS | FLGGLKWCSD | QSEIISNQYN | NEPSNIFEKI |
| DEENEANLLA | VLTETLDSLP | VDEDGLPSED | ALTDGDVTTD |
| NEASPSSMPD | GTPPPQEAEE | PSLLKKLLLA | PANTQLSYNE |
| CSGLSTQNHA | NHNHRIRTNP | AIVKTENSWS | NKAKSICQQQ |
| KPQRRPCSEL | LKYLTTNDDP | PHTKPTENRN | SSRDKCTSKK |
| KSHTQSQSQH | LQAKPTTLSL | PLTPESPNDP | KGSPFENKTI |
| ERTLSVELSG | TAGLaPPTTP | PHKANQDNPF | RASPKLKSSC |
| KTVVPPPSKK | PRYSESSGTQ | GNNSTKKGPE | QSELYAQLSK |
| SSVLTGGHEE | RKTKRPSLRL | FGDHDYCQSI | NSKTEILINI |
| SQELQDSRQL | ENKDVSSDWQ | GQICSSTDSD | QCYLRETLEA |
| SKQVSPCSTR | KQLQDQEIRA | ELNKHEGHPS | QAVFDDEADK |
| TGELRDSDFS | NEQFSKLPMF | INSGLAMDGL | FDDSEDESDK |
| LSYPWDGTQS | YSLFNVSPSC | SSFNSPCRDS | VSPPKSLFSQ |
| RPQRMRSRSR | SFSRHRSCSR | SPYSRSRSRS | PGSRSSSRSC |
| YYYESSHYRH | RTHRNSPLYV | RSRSRSPYSR | RPRYDSYEEY |
| QHERLKREEY | RREYEKRESE | RAKQRERQRQ | KAIEERRVIY |
| VGKIRPDTTR | TELRDRFEVF | GEIEECTVNL | RDDGDSYGFI |
| TYRYTCDAFA | ALENGYTLRR | SNETDFELYF | CGRKQFFKSN |
| YADLDSNSDD | FDPASTKSKY | DSLDFDSLLK | EAQRSLRR |

(SEQ ID NO: 3, T295A).

In some embodiments, there is a mutation in a Akt phosphorylation site. Akt phosphorylation of PGC-1α at S571 suppresses PGC-1α activity. Therefore, in some embodiments, there is a mutation to residue S571 of SEQ ID NO:1. In some embodiments, there is a mutation in an Akt phosphorylation site and a GSK3β-dependent ubiquitination site. Therefore, in some embodiments, the mutant PGC-1α comprises the amino acid sequence:

| | | | |
|---|---|---|---|
| MAWDMCNQDS | ESVWSDIECA | ALVGEDQPLC | PDLPELDLSE |
| LDVNDLDTDS | FLGGLKWCSD | QSEIISNQYN | NEPSNIFEKI |
| DEENEANLLA | VLTETLDSLP | VDEDGLPSED | ALTDGDVTTD |
| NEASPSSMPD | GTPPPQEAEE | PSLLKKLLLA | PANTQLSYNE |
| CSGLSTQNHA | NHNHRIRTNP | AIVKTENSWS | NKAKSICQQQ |
| KPQRRPCSEL | LKYLTTNDDP | PHTKPTENRN | SSRDKCTSKK |
| KSHTQSQSQH | LQAKPTTLSL | PLTPESPNDP | KGSPFENKTI |
| ERTLSVELSG | TAGLaPPTTP | PHKANQDNPF | RASPKLKSSC |
| KTVVPPPSKK | PRYSESSGTQ | GNNSTKKGPE | QSELYAQLSK |
| SSVLTGGHEE | RKTKRPSLRL | FGDHDYCQSI | NSKTEILINI |
| SQELQDSRQL | ENKDVSSDWQ | GQICSSTDSD | QCYLRETLEA |
| SKQVSPCSTR | KQLQDQEIRA | ELNKHFGHPS | QAVFDDEADK |
| TGELRDSDFS | NEQFSKLPMF | INSGLAMDGL | FDDSEDESDK |
| LSYPWDGTQS | YSLFNVSPSC | SSFNSPCRDS | VSPPKSLFSQ |
| RPQRMRSRSR | aFSRHRSCSR | SPYSRSRSRS | PGSRSSSRSC |
| YYYESSHYRH | RTHRNSPLYV | RSRSRSPYSR | RPRYDSYEEY |
| QHERLKREEY | RREYEKRESE | RAKQRERQRQ | KAIEERRVIY |
| VGKIRPDTTR | TELRDRFEVF | GEIEECTVNL | RDDGDSYGFI |
| TYRYTCDAFA | ALENGYTLRR | SNETDFELYF | CGRKQFFKSN |
| YADLDSNSDD | FDPASTKSKY | DSLDFDSLLK | EAQRSLRR |

(SEQ ID NO: 4, T295A, S571A).

In some embodiments, there is a mutation in a Clk-2 phosphorylation site. Clk-2 phosphorylation suppresses PGC-1α activity. Therefore, in some embodiments, there is a mutation to one or more of residues S567, S569, S573, S577, S579, S581, S599, S616, S624, S629, and S636 of SEQ ID NO:1. In some embodiments, there is a mutation to S571.

In some embodiments, there is a mutation in a S6K phosphorylation site. S6K phosphorylation suppresses PGC-1α activity. Therefore, in some embodiments, there is a mutation to residue S569 of SEQ ID NO:1.

Therefore, in some embodiments, the mutant PGC-1α has an amino acid mutation at T295, S569, S571, S573, S577, S579, S581, S599, S616, S624, S629, S636, or any combination thereof. In some embodiments, the mutant PGC-1α has an amino acid mutation at T295, S569, S573, S577, S579, S581, S599, S616, S624, S629, S636, or any combination thereof. In some embodiments, the mutant PGC-1α has a mutation at S571 and at least one more mutation. For example, in some embodiments, the mutant PGC-1α has a mutation at T295 and S571, which is referred to herein as a GA (GSK, Akt) mutation.

In some embodiments, the mutant PGC-1α has an Akt phosphorylation mutation at S571, an S6K phosphorylation mutation at S569, and a Clk-2 phosphorylation mutation at one or more of residues S569, S571, S573, S577, S579, S581, S599, S616, S624, S629, and S636, which is referred to herein as an ACS (Akt, Clk-2, S6K) mutation. Therefore, in some embodiments, the mutant PGC-1α comprises the amino acid sequence:

| | | | |
|---|---|---|---|
| MAWDMCNQDS | ESVWSDIECA | ALVGEDQPLC | PDLPELDLSE |
| LDVNDLDTDS | FLGGLKWCSD | QSEIISNQYN | NEPSNIFEKI |
| DEENEANLLA | VLTETLDSLP | VDEDGLPSED | ALTDGDVTTD |
| NEASPSSMPD | GTPPPQEAEE | PSLLKKLLLA | PANTQLSYNE |
| CSGLSTQNHA | NHNHRIRTNP | AIVKTENSWS | NKAKSICQQQ |
| KPQRRPCSEL | LKYLTTNDDP | PHTKPTENRN | SSRDKCTSKK |
| KSHTQSQSQH | LQAKPTTLSL | PLTPESPNDP | KGSPFENKTI |
| ERTLSVELSG | TAGLaPPTTP | PHKANQDNPF | RASPKLKSSC |
| KTVVPPPSKK | PRYSESSGTQ | GNNSTKKGPE | QSELYAQLSK |
| SSVLTGGHEE | RKTKRPSLRL | FGDHDYCQSI | NSKTEILINI |
| SQELQDSRQL | ENKDVSSDWQ | GQICSSTDSD | QCYLRETLEA |
| SKQVSPCSTR | KQLQDQEIRA | ELNKHEGHPS | QAVFDDEADK |
| TGELRDSDFS | NEQFSKLPMF | INSGLAMDGL | FDDSEDESDK |
| LSYPWDGTQS | YSLFNVSPSC | SSFNSPCRDS | VSPPKSLFSQ |
| RPQRMRaRaR | aFaRHRaCaR | aPYSRSRSRS | PGSRSSSRaC |
| YYYESSHYRH | RTHRNaPLYV | RSRaRSPYaR | RPRYDsYEEY |
| QHERLKREEY | RREYEKRESE | RAKQRERQRQ | KAIEERRVIY |
| VGKIRPDTTR | TELRDRFEVF | GEIEECTVNL | RDDGDSYGFI |
| TYRYTCDAFA | ALENGYTLRR | SNETDFELYF | CGRKQFFKSN |
| YADLDSNSDD | FDPASTKSKY | DSLDFDSLLK | EAQRSLRR |

(SEQ ID NO: 5, ACS).

In some embodiments, the mutation is any amino acid substitution that disrupts the post-translational modification, e.g. phosphorylation at the site, without disrupting PGC-1α coactivation, e.g. by affecting secondary or tertiary structure. In some embodiments, the amino acid is substituted with an alanine.

### NT-PGC-1α Proteins

NT-PGC-1α is a PGC-1α variant that contains an alternative splicing event between exons 6 and 7, which introduces a premature stop codon. This particular intronic sequence of the Pgc-1α gene, which is highly conserved in mammals, contains two distinct splicing acceptor sites. When the upstream acceptor site is favored, it yields a 270 aa protein named NT-PGC-1α that corresponds to the activation domain of PGC-1α1 (aa 11-80) and part of the repression domain (aa 180-403 in PGC-1α1; 180-267 in NT-PGC-1α). NT-PGC-1α lacks all the central and C-terminal PGC-1α1 protein modules, including the RS/RRM domains and the NLS. The absence of these sequences unmasks a nuclear export signal (NES) that under basal conditions localizes NT-PGC-1α mainly to the cytosol (90%). Although NT-PGC-1α proteins lack the C-terminal domain that in PGC-1α1 is required for interaction with the Mediator complex, they still coactivate PPARα and PPARγ transcriptional activity. However, this association is strictly ligand-dependent, probably because NT-PGC-1α lacks the amino acid sequences that in PGC-1α1 mediate ligand-independent coactivation of PPARs (aa 338-403 in PGC-1α1).

In some embodiments, the NT-PGC-1α isoform has the amino acid sequence:

| | | | |
|---|---|---|---|
| MAWDMCNQDS | ESVWSDIECA | ALVGEDQPLC | PDLPELDLSE |
| LDVNDLDTDS | FLGGLKWCSD | QSEIISNQYN | NEPSNIFEKI |
| DEENEANLLA | VLTETLDSLP | VDEDGLPSED | ALTDGDVTTD |
| NEASPSSMPD | GTPPPQEAEE | PSLLKKLLLA | PANTQLSYNE |
| CSGLSTQNHA | NHNHRIRTNP | AIVKTENSWS | NKAKSICQQQ |
| KPQRRPCSEL | LKYLTTNDDP | PHTKPTENRN | SSRDKCTSKK |
| KSHTQSQSQH | LQAKPTTLSL | PLTPESPNLF | L |

(SEQ ID NO: 2, NT-PGC-1a).

In some embodiments, the NT-PGC-1α isoform is a conservative variant of SEQ ID NO:2, or a conservative variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% sequence identity to SEQ ID NO:2. A conservative variant includes any amino acid substitution that does not affect the function of the polypeptide. Conservative substitutions are known in the art and can be predicted using routine methods.

In some embodiments, the NT-PGC-1α isoform has one or more mutations, such as mutations to a post-translational modification (PTM) site. For example, in some embodiments, there is a mutation in a Nuclear Export Sequence (NES). NES removes PGC-1α from the nucleus. Therefore, in some embodiments, there is a mutation to one or more of residues L29, L33, L36, L38, L92, L96, L99, and V101 of SEQ ID NO:2.

In some embodiments, there is a mutation in a GCN5 acetylation site. GCN5 suppresses activity. Therefore, in some embodiments, there is a mutation to one or more of residues K78, K145, K184, and K254 of SEQ ID NO:2.

In some embodiments, there is a mutation in a SUMOylation site. Sumoylation suppresses activity. Therefore, in some embodiments, there is a mutation to one or more of residues V183, T185, and E186 of SEQ ID NO:2

In some embodiments, there is a mutation in a PKA phosphorylation site. Phosphorylation at these sites increases PGC-1α activity, mutating to aspartic acid mimics phosphorylation. Therefore, in some embodiments, there is a mutation to one or more of residues S195, S242, and T257 of SEQ ID NO:2

In some embodiments, there is a mutation in a p38MAPK phosphorylation site. Phosphorylation at these sites increases PGC-1α activity, mutating to aspartic acid mimics phosphorylation. Therefore, in some embodiments, there is a mutation to one or more of residues T263, S266, and L269 of SEQ ID NO:2.

In some embodiments, the NT-PGC-1α isoform has a mutation in an NES, a GCN5 site, a SUMOylation site, a PKA site, and a p38MAPK site.

In some embodiments, the NT-PGC-1α isoform has a mutation in a GCN5 site, a SUMOylation site, a PKA site, and a p38MAPK site. In some embodiments, the NT-PGC-1α isoform has a mutation in an NES, a SUMOylation site, a PKA site, and a p38MAPK site. In some embodiments, the NT-PGC-1α isoform has a mutation in an NES, a GCN5 site, a SUMOylation site, and a p38MAPK site. In some embodiments, the NT-PGC-1α isoform has a mutation in an NES, a GCN5 site, a SUMOylation site, and a PKA site.

In some embodiments, the NT-PGC-1α isoform has a mutation in a SUMOylation site, a PKA site, and a p38MAPK site. In some embodiments, the NT-PGC-1α isoform has a mutation in a GCN5 site, a PKA site, and a p38MAPK site. In some embodiments, the NT-PGC-1α isoform has a mutation in a GCN5 site, a SUMOylation site, and a p38MAPK site. In some embodiments, the NT-PGC-1α isoform has a mutation in a GCN5 site, a SUMOylation site, and a PKA site. In some embodiments, the NT-PGC-1α isoform has a mutation in a SUMOylation site, a PKA site, and a p38MAPK site. In some embodiments, the NT-PGC-1α isoform has a mutation in an NES, a PKA site, and a p38MAPK site. In some embodiments, the NT-PGC-1α isoform has a mutation in an NES, a SUMOylation site, and a p38MAPK site. In some embodiments, the NT-PGC-1α isoform has a mutation in an NES, a SUMOylation site, and a PKA site. In some embodiments, the NT-PGC-1α isoform has a mutation in a GCN5 site, a PKA site, and a p38MAPK site. In some embodiments, the NT-PGC-1α isoform has a mutation in an NES, a PKA site, and a p38MAPK site. In some embodiments, the NT-PGC-1α isoform has a mutation in an NES, a GCN5 site, and a p38MAPK site. In some embodiments, the NT-PGC-1α isoform has a mutation in an NES, a GCN5 site, and a PKA site. In some embodiments, the NT-PGC-1α isoform has a mutation in a GCN5 site, a SUMOylation site, and a p38MAPK site. In some embodiments, the NT-PGC-1α isoform has a mutation in an NES, a SUMOylation site, and a p38MAPK site. In some embodiments, the NT-PGC-1α isoform has a mutation in an NES, a GCN5 site, and a p38MAPK site. In some embodiments, the NT-PGC-1α isoform has a mutation in an NES, a GCN5 site, and a SUMOylation site. In some embodiments, the NT-PGC-1α isoform has a mutation in a GCN5 site, a SUMOylation site, and a PKA site. In some embodiments, the NT-PGC-1α isoform has a mutation in an NES, a SUMOylation site, and a PKA site. In some embodiments, the NT-PGC-1α isoform has a mutation in an NES, a GCN5 site, and a PKA site. In some embodiments, the NT-PGC-1α isoform has a mutation in an NES, a GCN5 site, and a SUMOylation site.

In some embodiments, the NT-PGC-1α isoform has a mutation in an NES and a GCN5 site. In some embodiments, the NT-PGC-1α isoform has a mutation in an NES and a SUMOylation site. In some embodiments, the NT-PGC-1α isoform has a mutation in an NES and a PKA site. In some embodiments, the NT-PGC-1α isoform has a mutation in an NES and a p38MAPK site. In some embodiments, the NT-PGC-1α isoform has a mutation in a GCN5 site and a SUMOylation site. In some embodiments, the NT-PGC-1α isoform has a mutation in a GCN5 site and a PKA site. In some embodiments, the NT-PGC-1α isoform has a mutation in a GCN5 site and a p38MAPK site. In some embodiments, the NT-PGC-1α isoform has a mutation in a SUMOylation site and a PKA site. In some embodiments, the NT-PGC-1α isoform has a mutation in a SUMOylation site and a p38MAPK site. In some embodiments, the NT-PGC-1α isoform has a mutation in a PKA site and p38MAPK site.

Therefore, in some embodiments, the NT-PGC-1α mutant has the amino acid sequence:

| | | | |
|---|---|---|---|
| MAWDMCNQDS | ESVWSDIECA | ALVGEDQPaC | PDaPEaDaSE |
| LDVNDLDTDS | FLGGLKWCSD | QSEIISNQYN | NEPSNIFEal |
| DEENEANLLA | VLTETLDSLP | VDEDGLPSED | ALTDGDVTTD |
| NEASPSSMPD | GTPPPQEAEE | PSLLaKLLLA | PANTQLSYNE |
| CSGLSTQNHA | NHNHRIRTNP | AIVaTENSWS | NKAKSICQQQ |
| KPQRRPCSEL | LKYLTTNDDP | PHTKPTENRN | SSRDKCTSKK |
| KSHTQSQSQH | LQAaPTTLSL | PLTPESPNLF | L |

(SEQ ID NO: 6, mNT - NES1/GCN5).

Therefore, in some embodiments, the NT-PGC-1α mutant has the amino acid sequence:

| | | | |
|---|---|---|---|
| MAWDMCNQDS | ESVWSDIECA | ALVGEDQPaC | PDaPEaDaSE |
| LDVNDLDTDS | FLGGLKWCSD | QSEIISNQYN | NEPSNIFEaI |
| DEENEANLLA | VLTETaDSaP | aDEDGLPSED | ALTDGDVTTD |
| NEASPSSMPD | GTPPPQEAEE | PSLLaKLLLA | PANTQLSYNE |
| CSGLSTQNHA | NHNHRIRTNP | AIVaTENSWS | NKAKSICQQQ |
| KPQRRPCSEL | LKYLTTNDDP | PHTKPTENRN | SSRDKCTSKK |
| KSHTQSQSQH | LQAaPTTLSL | PLTPESPNLF | L |

(SEQ ID NO: 7, mNT2 - NES1/NES2/GCN5).

Therefore, in some embodiments, the NT-PGC-1α mutant has the amino acid sequence:

| | | | |
|---|---|---|---|
| MAWDMCNQDS | ESVWSDIECA | ALVGEDQPaC | PDaPEaDaSE |
| LDVNDLDTDS | FLGGLKWCSD | QSEIISNQYN | NEPSNIFEaI |
| DEENEANLLA | VLTETaDSaP | aDEDGLPSED | ALTDGDVTTD |
| NEASPSSMPD | GTPPPQEAEE | PSLLaKLLLA | PANTQLSYNE |
| CSGLSTQNHA | NHNHRIRTNP | AIVaTENSWS | NKAKdICQQQ |
| KPQRRPCSEL | LKYLTTNDDP | PHTKPTENRN | SSRDKCTSKK |
| KdHTQSQSQH | LQAaPTdLSL | PLdPEdPNdF | L |

(SEQ ID NO: 8, mNT3 - NES1/NES2/GCN5/
PKA/p38MAPK).

Therefore, in some embodiments, the NT-PGC-1α mutant has the amino acid sequence:

| | | | |
|---|---|---|---|
| MAWDMCNQDS | ESVWSDIECA | ALVGEDQPaC | PDaPEaDaSE |
| LDVNDLDTDS | FLGGLKWCSD | QSEIISNQYN | NEPSNIFEKI |
| DEENEANLLA | VLTETaDSaP | aDEDGLPSED | ALTDGDVTTD |
| NEASPSSMPD | GTPPPQEAEE | PSLLKKLLLA | PANTQLSYNE |
| CSGLSTQNHA | NHNHRIRTNP | AIVKTENSWS | NKAKdICQQQ |
| KPQRRPCSEL | LKYLTTNDDP | PHTKPTENRN | SSRDKCTSKK |
| KdHTQSQSQH | LQAKPTdLSL | PLTPESPNLF | L |

(SEQ ID NO: 9, mNT4 - NES1/NES2/PKA).

Therefore, in some embodiments, the NT-PGC-1α mutant has the amino acid sequence:

| | | | |
|---|---|---|---|
| MAWDMCNQDS | ESVWSDIECA | ALVGEDQPaC | PDaPEaDaSE |
| LDVNDLDTDS | FLGGLKWCSD | QSEIISNQYN | NEPSNIFEKI |
| DEENEANLLA | VLTETaDSaP | aDEDGLPSED | ALTDGDVTTD |
| NEASPSSMPD | GTPPPQEAEE | PSLLKKLLLA | PANTQLSYNE |
| CSGLSTQNHA | NHNHRIRTNP | AIVKTENSWS | NKAKSICQQQ |
| KPQRRPCSEL | LKYLTTNDDP | PHTKPTENRN | SSRDKCTSKK |
| KSHTQSQSQH | LQAKPTTLSL | PLdPEdPNdF | L |

(SEQ ID NO: 10, mNT5 - NES1/NES2/p38MAPK)

Therefore, in some embodiments, the NT-PGC-1α mutant has the amino acid sequence:

| | | | |
|---|---|---|---|
| MAWDMCNQDS | ESVWSDIECA | ALVGEDQPaC | PDaPEaDaSE |
| LDVNDLDTDS | FLGGLKWCSD | QSEIISNQYN | NEPSNIFEKI |
| DEENEANLLA | VLTETLDSLP | VDEDGLPSED | ALTDGDVTTD |
| NEASPSSMPD | GTPPPQEAEE | PSLLKKLLLA | PANTQLSYNE |
| CSGLSTQNHA | NHNHRIRTNP | AIVKTENSWS | NKAKdICQQQ |
| KPQRRPCSEL | LKYLTTNDDP | PHTKPTENRN | SSRDKCTSKK |
| KdHTQSQSQH | LQAKPTdLSL | PLTPESPNLF | L |

(SEQ ID NO: 11), mNT6 - NES1/PKA).

Therefore, in some embodiments, the NT-PGC-1α mutant has the amino acid sequence:

MAWDMCNQDSESVWSDIECAALVGEDQPaCPDaPEaDaSELDVNDLDTDS
FLGGLKWCSDQSEIISNQYNNEPSNIFEKIDEENEANLLAVLTETLDSLP
VDEDGLPSFDALTDGDVTTDNEASPSSMPDGTPPPQEAEEPSLLKKLLLA
PANTQLSYNECSGLSTQNHANHNHRIRTNPAIVKTENSWSNKAKSICQQQ
KPQRRPCSELLKYLTTNDDPPHTKPTENRNSSRDKCTSKKKSHTQSQSQH
LQAKPTTLSLPLdPEdPNdFL (SEQ ID NO: 12, mNT7 -
NES1/p38MAPK).

Therefore, in some embodiments, the NT-PGC-1α mutant has the amino acid sequence:

| | | | |
|---|---|---|---|
| MAWDMCNQDS | ESVWSDIECA | ALVGEDQPLC | PDLPELDLSE |
| LDVNDLDTDS | FLGGLKWCSD | QSEIISNQYN | NEPSNIFEKI |
| DEENEANLLA | VLTETLDSLP | VDEDGLPSED | ALTDGDVTTD |
| NEASPSSMPD | GTPPPQEAEE | PSLLKKLLLA | PANTQLSYNE |
| CSGLSTQNHA | NHNHRIRTNP | AIVKTENSWS | NKAKdICQQQ |
| KPQRRPCSEL | LKYLTTNDDP | PHTKPTENRN | SSRDKCTSKK |
| KdHTQSQSQH | LQAKPTdLSL | PLTPESPNLF | L |

(SEQ ID NO: 13, mNT8 - PKA).

Therefore, in some embodiments, the NT-PGC-1α mutant has the amino acid sequence:

| | | | |
|---|---|---|---|
| MAWDMCNQDS | ESVWSDIECA | ALVGEDQPLC | PDLPELDLSE |
| LDVNDLDTDS | FLGGLKWCSD | QSEIISNQYN | NEPSNIFEKI |
| DEENEANLLA | VLTETLDSLP | VDEDGLPSED | ALTDGDVTTD |
| NEASPSSMPD | GTPPPQEAEE | PSLLKKLLLA | PANTQLSYNE |
| CSGLSTQNHA | NHNHRIRTNP | AIVKTENSWS | NKAKSICQQQ |
| KPQRRPCSEL | LKYLTTNDDP | PHTKPTENRN | SSRDKCTSKK |
| KSHTQSQSQH | LQAKPTTLSL | PLdPEdPNdF | L |

(SEQ ID NO: 14, mNT9 - p38MAPK)

Therefore, in some embodiments, the NT-PGC-1α mutant has the amino acid sequence:

| | | | |
|---|---|---|---|
| MAWDMCNQDS | ESVWSDIECA | ALVGEDQPLC | PDLPELDLSE |
| LDVNDLDTDS | FLGGLKWCSD | QSEIISNQYN | NEPSNIFEaI |
| DEENEANLLA | VLTETLDSLP | VDEDGLPSED | ALTDGDVTTD |
| NEASPSSMPD | GTPPPQEAEE | PSLLaKLLLA | PANTQLSYNE |
| CSGLSTQNHA | NHNHRIRTNP | AIVaTENSWS | NKAKSICQQQ |
| KPQRRPCSEL | LKYLTTNDDP | PHTKPTENRN | SSRDKCTSKK |
| KSHTQSQSQH | LQAaPTTLSL | PLTPESPNLF | L |

(SEQ ID NO: 15, mNT10 - GCN5).

In some embodiments, the mutation is any amino acid substitution that disrupts the post-translational modification, e.g. phosphorylation at the site, without disrupting PGC-1α coactivation, e.g. by affecting secondary or tertiary structure. In some embodiments, the amino acid is substituted with an alanine.

Here again, in some embodiments, the mutation is any amino acid substitution that disrupts the post-translational modification, e.g. phosphorylation at the site, without disrupting PGC-1α coactivation, e.g. by affecting secondary or tertiary structure. In some embodiments, the amino acid is substituted with an alanine.

### CAR Polypeptides

The disclosed methods can be used to produce chimeric antigen receptor (CAR) T cells containing CAR polypeptides. A CAR polypeptide is generally made up of three domains: an ectodomain, a transmembrane domain, and an endodomain. The ectodomain is responsible for antigen recognition. It also optionally contains a signal peptide (SP) so that the CAR can be glycosylated and anchored in the cell membrane of the immune effector cell. The transmembrane domain (TD), is as its name suggests, connects the ectodomain to the endodomain and resides within the cell membrane when expressed by a cell. The endodomain is the business end of the CAR that transmits an activation signal to the immune effector cell after antigen recognition. For example, the endodomain can contain an intracellular signaling domain (ISD) and optionally a co-stimulatory signaling region (CSR). CAR polypeptides generally incorporate an antigen recognition domain from the single-chain variable fragments (scFv) of a monoclonal antibody (mAb) with transmembrane signaling motifs involved in lymphocyte activation (Sadelain M, et al. Nat Rev Cancer 2003 3:35-45).

A “signaling domain (SD)” generally contains immunoreceptor tyrosine-based activation motifs (ITAMs) that activate a signaling cascade when the ITAM is phosphorylated. The term “co-stimulatory signaling region (CSR)” refers to intracellular signaling domains from costimulatory protein receptors, such as CD28, 41BB, and ICOS, that are able to enhance T-cell activation by T-cell receptors.

Additional CAR constructs are described, for example, in Fresnak A D, et al. Engineered T cells: the promise and challenges of cancer immunotherapy. Nat Rev Cancer. 2016 Aug. 23; 16(9):566-81, which is incorporated by reference in its entirety for the teaching of these CAR models.

For example, the CAR can be a TRUCK, Universal CAR, Self-driving CAR, Armored CAR, Self-destruct CAR, Conditional CAR, Marked CAR, TenCAR, Dual CAR, or sCAR.

CAR T cells engineered to be resistant to immunosuppression (Armored CARs) may be genetically modified to no longer express various immune checkpoint molecules (for example, cytotoxic T lymphocyte-associated antigen 4 (CTLA4) or programmed cell death protein 1 (PD1)), with an immune checkpoint switch receptor, or may be administered with a monoclonal antibody that blocks immune checkpoint signaling.

A self-destruct CAR may be designed using RNA delivered by electroporation to encode the CAR. Alternatively, inducible apoptosis of the T cell may be achieved based on ganciclovir binding to thymidine kinase in gene-modified lymphocytes or the more recently described system of activation of human caspase 9 by a small-molecule dimerizer.

A conditional CAR T cell is by default unresponsive, or switched ‘off’, until the addition of a small molecule to complete the circuit, enabling full transduction of both signal 1 and signal 2, thereby activating the CAR T cell. Alternatively, T cells may be engineered to express an adaptor-specific receptor with affinity for subsequently administered secondary antibodies directed at target antigen.

A tandem CAR (TanCAR) T cell expresses a single CAR consisting of two linked single-chain variable fragments (scFvs) that have different affinities fused to intracellular co-stimulatory domain(s) and a CD3ζ domain. TanCAR T cell activation is achieved only when target cells co-express both targets.

A dual CART cell expresses two separate CARs with different ligand binding targets; one CAR includes only the CD3ζ domain and the other CAR includes only the co-stimulatory domain(s). Dual CAR T cell activation requires co-expression of both targets.

A safety CAR (sCAR) consists of an extracellular scFv fused to an intracellular inhibitory domain. sCAR T cells co-expressing a standard CAR become activated only when encountering target cells that possess the standard CAR target but lack the sCAR target.

The antigen recognition domain of the disclosed CAR is usually an scFv. There are however many alternatives. An antigen recognition domain from native T-cell receptor (TCR) alpha and beta single chains have been described, as have simple ectodomains (e.g. CD4 ectodomain to recognize HIV infected cells) and more exotic recognition components such as a linked cytokine (which leads to recognition of cells bearing the cytokine receptor). In fact almost anything that binds a given target with high affinity can be used as an antigen recognition region.

The endodomain is the business end of the CAR that after antigen recognition transmits a signal to the immune effector cell, activating at least one of the normal effector functions of the immune effector cell. Effector function of a T cell, for example, may be cytolytic activity or helper activity including the secretion of cytokines. Therefore, the endodomain may comprise the “intracellular signaling domain” of a T cell receptor (TCR) and optional co-receptors. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal.

Cytoplasmic signaling sequences that regulate primary activation of the TCR complex that act in a stimulatory manner may contain signaling motifs which are known as immunoreceptor tyrosine-based activation motifs (ITAMs). Examples of ITAM containing cytoplasmic signaling sequences include those derived from CD8, CD3ζ, CD3ζ, CD3γ, CD3ε, CD32 (Fc gamma RIIa), DAP10, DAP12, CD79a, CD79b, FcγRIγ, FcγRIIIγ, FcεRIβ (FCERIB), and FcεRIγ (FCERIG).

In particular embodiments, the intracellular signaling domain is derived from CD3 zeta (CD3ζ) (TCR zeta, GenBank accno. BAG36664.1). T-cell surface glycoprotein CD3 zeta (CD3ζ) chain, also known as T-cell receptor T3 zeta chain or CD247 (Cluster of Differentiation 247), is a protein that in humans is encoded by the CD247 gene.

First-generation CARs typically had the intracellular domain from the CD3ζ chain, which is the primary transmitter of signals from endogenous TCRs. Second-generation CARs add intracellular signaling domains from various costimulatory protein receptors (e.g., CD28, 41BB, ICOS) to the endodomain of the CAR to provide additional signals to the T cell. More recent, third-generation CARs combine multiple signaling domains to further augment potency. T cells grafted with these CARs have demonstrated improved expansion, activation, persistence, and tumor-eradicating efficiency independent of costimulatory receptor/ligand interaction (Imai C, et al. Leukemia 2004 18:676-84; Maher J, et al. Nat Biotechnol 2002 20:70-5).

For example, the endodomain of the CAR can be designed to comprise the CD3ζ signaling domain by itself or combined with any other desired cytoplasmic domain(s) useful in the context of the CAR of the invention. For example, the cytoplasmic domain of the CAR can comprise a CD3ζ chain portion and a costimulatory signaling region. The costimulatory signaling region refers to a portion of the CAR comprising the intracellular domain of a costimulatory molecule. A costimulatory molecule is a cell surface molecule other than an antigen receptor or their ligands that is required for an efficient response of lymphocytes to an antigen. Examples of such molecules include CD27, CD28, 4-1BB (CD137), OX40, CD30, CD40, ICOS, lymphocyte function-associated antigen-1 (LFA-1), CD2, CD7, LIGHT, NKG2C, B7-H3, and a ligand that specifically binds with CD123, CD8, CD4, b2c, CD80, CD86, DAP10, DAP12, MyD88, BTNL3, and NKG2D. Thus, while the CAR is exemplified primarily with CD28 as the co-stimulatory signaling element, other costimulatory elements can be used alone or in combination with other co-stimulatory signaling elements.

In some embodiments, the CAR comprises a hinge sequence. A hinge sequence is a short sequence of amino acids that facilitates antibody flexibility (see, e.g., Woof et al., Nat. Rev. Immunol., 4(2): 89-99 (2004)). The hinge sequence may be positioned between the antigen recognition moiety (e.g., scFv) and the transmembrane domain. The hinge sequence can be any suitable sequence derived or obtained from any suitable molecule. In some embodiments, for example, the hinge sequence is derived from a CD8a molecule or a CD28 molecule.

The transmembrane domain may be derived either from a natural or from a synthetic source. Where the source is natural, the domain may be derived from any membrane-bound or transmembrane protein. For example, the transmembrane region may be derived from (i.e. comprise at least the transmembrane region(s) of) the alpha, beta or zeta chain of the T-cell receptor, CD28, CD3 epsilon, CD45, CD4, CDS, CD8 (e.g., CD8 alpha, CD8 beta), CD9, CD16, CD22, CD33, CD37, CD64, CD80, CD86, CD134, CD137, or CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7R α, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, and PAG/Cbp. Alternatively the transmembrane domain may be synthetic, in which case it will comprise predominantly hydrophobic residues such as leucine and valine. In some cases, a triplet of phenylalanine, tryptophan and valine will be found at each end of a synthetic transmembrane domain. A short oligo- or polypeptide linker, such as between 2 and 10 amino acids in length, may form the linkage between the transmembrane domain and the endoplasmic domain of the CAR.

In some embodiments, the CAR has more than one transmembrane domain, which can be a repeat of the same transmembrane domain, or can be different transmembrane domains.

In some embodiments, the CAR is a multi-chain CAR, as described in WO2015/039523, which is incorporated by reference for this teaching. A multi-chain CAR can comprise separate extracellular ligand binding and signaling domains in different transmembrane polypeptides. The signaling domains can be designed to assemble in juxtamembrane position, which forms flexible architecture closer to natural receptors, that confers optimal signal transduction. For example, the multi-chain CAR can comprise a part of an FCERI alpha chain and a part of an FCERI beta chain such that the FCERI chains spontaneously dimerize together to form a CAR.

In some embodiments, the antigen recognition domain is single chain variable fragment (scFv) antibody. The affinity/specificity of an scFv is driven in large part by specific sequences within complementarity determining regions (CDRs) in the heavy (V_{H}) and light (V_{L}) chain. Each V_{H} and V_{L} sequence will have three CDRs (CDR1, CDR2, CDR3).

In some embodiments, the antigen recognition domain is derived from natural antibodies, such as monoclonal antibodies. In some cases, the antibody is human. In some cases, the antibody has undergone an alteration to render it less immunogenic when administered to humans. For example, the alteration comprises one or more techniques selected from the group consisting of chimerization, humanization, CDR-grafting, deimmunization, and mutation of framework amino acids to correspond to the closest human germline sequence.

Also disclosed are bi-specific CARs that target two antigens. Also disclosed are CARs designed to work only in conjunction with another CAR that binds a different antigen. For example, in these embodiments, the endodomain of the disclosed CAR can contain only a signaling domain (SD) or a co-stimulatory signaling region (CSR), but not both. The second CAR (or endogenous T-cell) provides the missing signal if it is activated. For example, if the disclosed CAR contains an SD but not a CSR, then the immune effector cell containing this CAR is only activated if another CAR (or T-cell) containing a CSR binds its respective antigen. Likewise, if the disclosed CAR contains a CSR but not a SD, then the immune effector cell containing this CAR is only activated if another CAR (or T-cell) containing an SD binds its respective antigen.

### Immune Effector Cells

Also disclosed are immune effector cells that are engineered to express the disclosed CARs. These cells are preferably obtained from the subject to be treated (i.e. are autologous). However, in some embodiments, immune effector cell lines or donor effector cells (allogeneic) are used. In still other embodiments, the immune effect cells are not HLA-matched. Immune effector cells can be obtained from a number of sources, including peripheral blood mononuclear cells, bone marrow, lymph node tissue, cord blood, thymus tissue, tissue from a site of infection, ascites, pleural effusion, spleen tissue, and tumors. Immune effector cells can be obtained from blood collected from a subject using any number of techniques known to the skilled artisan, such as Ficoll™ separation. For example, cells from the circulating blood of an individual may be obtained by apheresis. In some embodiments, immune effector cells are isolated from peripheral blood lymphocytes by lysing the red blood cells and depleting the monocytes, for example, by centrifugation through a PERCOLL™ gradient or by counterflow centrifugal elutriation. A specific subpopulation of immune effector cells can be further isolated by positive or negative selection techniques. For example, immune effector cells can be isolated using a combination of antibodies directed to surface markers unique to the positively selected cells, e.g., by incubation with antibody-conjugated beads for a time period sufficient for positive selection of the desired immune effector cells. Alternatively, enrichment of immune effector cells population can be accomplished by negative selection using a combination of antibodies directed to surface markers unique to the negatively selected cells.

In some embodiments, the immune effector cells comprise any leukocyte involved in defending the body against infectious disease and foreign materials. For example, the immune effector cells can comprise lymphocytes, monocytes, macrophages, dendritic cells, mast cells, neutrophils, basophils, eosinophils, or any combinations thereof. For example, the immune effector cells can comprise T lymphocytes.

T cells or T lymphocytes can be distinguished from other lymphocytes, such as B cells and natural killer cells (NK cells), by the presence of a T-cell receptor (TCR) on the cell surface. They are called T cells because they mature in the thymus (although some also mature in the tonsils). There are several subsets of T cells, each with a distinct function.

T helper cells (T_{H} cells) assist other white blood cells in immunologic processes, including maturation of B cells into plasma cells and memory B cells, and activation of cytotoxic T cells and macrophages. These cells are also known as CD4+ T cells because they express the CD4 glycoprotein on their surface. Helper T cells become activated when they are presented with peptide antigens by MHC class II molecules, which are expressed on the surface of antigen-presenting cells (APCs). Once activated, they divide rapidly and secrete small proteins called cytokines that regulate or assist in the active immune response. These cells can differentiate into one of several subtypes, including T_{H}1, T_{H}2, T_{H}3, T_{H}17, T_{H}9, or T_{FH}, which secrete different cytokines to facilitate a different type of immune response.

Cytotoxic T cells (T_{C} cells, or CTLs) destroy virally infected cells and tumor cells, and are also implicated in transplant rejection. These cells are also known as CD8⁺ T cells since they express the CD8 glycoprotein at their surface. These cells recognize their targets by binding to antigen associated with MHC class I molecules, which are present on the surface of all nucleated cells. Through IL-10, adenosine and other molecules secreted by regulatory T cells, the CD8+ cells can be inactivated to an anergic state, which prevents autoimmune diseases.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory cells may be either CD4⁺ or CD8⁺. Memory T cells typically express the cell surface protein CD45RO.

Regulatory T cells (T_{reg} cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus. Two major classes of CD4⁺ T_{reg} cells have been described—naturally occurring T_{reg} cells and adaptive T_{reg} cells.

Natural killer T (NKT) cells (not to be confused with natural killer (NK) cells) bridge the adaptive immune system with the innate immune system. Unlike conventional T cells that recognize peptide antigens presented by major histocompatibility complex (MHC) molecules, NKT cells recognize glycolipid antigen presented by a molecule called CD1d.

In some embodiments, the T cells comprise a mixture of CD4⁺ cells. In other embodiments, the T cells are enriched for one or more subsets based on cell surface expression. For example, in some cases, the T comprise are cytotoxic CD8⁺ T lymphocytes. In some embodiments, the T cells comprise γδ T cells, which possess a distinct T-cell receptor (TCR) having one γ chain and one δ chain instead of α and β chains.

Natural-killer (NK) cells are CD56⁺CD3⁻ large granular lymphocytes that can kill virally infected and transformed cells, and constitute a critical cellular subset of the innate immune system (Godfrey J, et al. Leuk Lymphoma 2012 53:1666-1676). Unlike cytotoxic CD8⁺ T lymphocytes, NK cells launch cytotoxicity against tumor cells without the requirement for prior sensitization, and can also eradicate MHC-I-negative cells (Narni-Mancinelli E, et al. Int Immunol 2011 23:427-431). NK cells are safer effector cells, as they may avoid the potentially lethal complications of cytokine storms (Morgan R A, et al. Mol Ther 2010 18:843-851), tumor lysis syndrome (Porter D L, et al. N Engl J Med 2011 365:725-733), and on-target, off-tumor effects. Although NK cells have a well-known role as killers of cancer cells, and NK cell impairment has been extensively documented as crucial for progression of MM (Godfrey J, et al. Leuk Lymphoma 2012 53:1666-1676; Fauriat C, et al. Leukemia 2006 20:732-733), the means by which one might enhance NK cell-mediated anti-MM activity has been largely unexplored prior to the disclosed CARs.

Epstein-Barr virus (EBV)-induced lymphoproliferative diseases (EBV-LPDs) are a significant cause of morbidity and mortality for recipients of allogeneic hematopoietic cell transplantation (HCT), particularly in those who have received certain T-cell reactive Abs to prevent or treat GVHD. Prophylaxis and treatment by the adoptive transfer of EBV-specific T cells and the subsequent long-term restoration of immunity against EBV-associated lymphoproliferation have provided positive outcomes in the management of this uniformly fatal complication of bone marrow transfer. Therefore, in some embodiments, the disclosed immune effector cells expressing the CARs of the present invention are allogeneic or autologous EBV-specific cytotoxic T lymphocytes (CTLs). For example, this can involve isolating PBMCs from of an autologous or allogenic donor and enriching them for T cells by depletion of monocytes and NK cells. For example, the donor can be an EBV-seropositive donor. These T cells can then be stimulated with autologous EBV-seropostive or transformed lymphocytes. EBV antigens include latent membrane protein (LMP) and EBV nuclear antigen (EBNA) proteins, such as LMP-1, LMP-2A, and LMP-2B and EBNA-1, EBNA-2, EBNA-3A, EBNA-3B, EBNA-3C and EBNA-LP. These methods are described, for example, in Wilkie et al., J. Immunother. 27(4):309-316 (2004), Barker et al., Blood 2010 116(23):5045-49; Doubrovina, et al., Blood 2012 119(11):2644-56; Koehne, et al. Blood 2002 99(5):1730-40; and Smith et al. Cancer Res 2012 72(5):1116-25, which are incorporated by reference for these teachings.

### Therapeutic Methods

Immune effector cells expressing the disclosed CARs can elicit an anti-tumor immune response against TAA-expressing cancer cells. The anti-tumor immune response elicited by the disclosed CAR-modified immune effector cells may be an active or a passive immune response. In addition, the CAR-mediated immune response may be part of an adoptive immunotherapy approach in which CAR-modified immune effector cells induce an immune response specific to TAA.

Adoptive transfer of immune effector cells expressing chimeric antigen receptors is a promising anti-cancer therapeutic. Following the collection of a patient's immune effector cells, the cells may be genetically engineered to express the disclosed CARs, then infused back into the patient.

The disclosed CAR-modified immune effector cells may be administered either alone, or as a pharmaceutical composition in combination with diluents and/or with other components such as IL-2, IL-15, or other cytokines or cell populations. Briefly, pharmaceutical compositions may comprise a target cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions for use in the disclosed methods are in some embodiments formulated for intravenous administration. Pharmaceutical compositions may be administered in any manner appropriate treat MM. The quantity and frequency of administration will be determined by such factors as the condition of the patient, and the severity of the patient's disease, although appropriate dosages may be determined by clinical trials.

When “an immunologically effective amount”, “an anti-tumor effective amount”, “an tumor-inhibiting effective amount”, or “therapeutic amount” is indicated, the precise amount of the compositions of the present invention to be administered can be determined by a physician with consideration of individual differences in age, weight, tumor size, extent of infection or metastasis, and condition of the patient (subject). It can generally be stated that a pharmaceutical composition comprising the T cells described herein may be administered at a dosage of 10⁴ to 10⁹ cells/kg body weight, such as 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

In certain embodiments, it may be desired to administer activated T cells to a subject and then subsequently re-draw blood (or have an apheresis performed), activate T cells therefrom according to the disclosed methods, and reinfuse the patient with these activated and expanded T cells. This process can be carried out multiple times every few weeks. In certain embodiments, T cells can be activated from blood draws of from 10 cc to 400 cc. In certain embodiments, T cells are activated from blood draws of 20 cc, 30 cc, 40 cc, 50 cc, 60 cc, 70 cc, 80 cc, 90 cc, or 100 cc. Using this multiple blood draw/multiple reinfusion protocol may serve to select out certain populations of T cells.

The administration of the disclosed compositions may be carried out in any convenient manner, including by injection, transfusion, or implantation. The compositions described herein may be administered to a patient subcutaneously, intradermally, intratumorally, intranodally, intramedullary, intramuscularly, by intravenous (i.v.) injection, or intraperitoneally. In some embodiments, the disclosed compositions are administered to a patient by intradermal or subcutaneous injection. In some embodiments, the disclosed compositions are administered by i.v. injection. The compositions may also be injected directly into a tumor, lymph node, or site of infection.

In certain embodiments, the disclosed CAR-modified immune effector cells are administered to a patient in conjunction with (e.g., before, simultaneously or following) any number of relevant treatment modalities, including but not limited to thalidomide, dexamethasone, bortezomib, and lenalidomide. In further embodiments, the CAR-modified immune effector cells may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. In some embodiments, the CAR-modified immune effector cells are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the cell compositions of the present invention are administered following B-cell ablative therapy such as agents that react with CD20, e.g., Rituxan. For example, in some embodiments, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

The cancer of the disclosed methods can be any TAA-expressing cell in a subject undergoing unregulated growth, invasion, or metastasis. In some aspects, the cancer can be any neoplasm or tumor for which radiotherapy is currently used. Alternatively, the cancer can be a neoplasm or tumor that is not sufficiently sensitive to radiotherapy using standard methods. Thus, the cancer can be a sarcoma, lymphoma, leukemia, carcinoma, blastoma, or germ cell tumor. A representative but non-limiting list of cancers that the disclosed compositions can be used to treat include lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, endometrial cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, and pancreatic cancer.

The disclosed CARs can be used in combination with any compound, moiety or group which has a cytotoxic or cytostatic effect. Drug moieties include chemotherapeutic agents, which may function as microtubulin inhibitors, mitosis inhibitors, topoisomerase inhibitors, or DNA intercalators, and particularly those which are used for cancer therapy.

The disclosed CARs can be used in combination with a checkpoint inhibitor. The two known inhibitory checkpoint pathways involve signaling through the cytotoxic T-lymphocyte antigen-4 (CTLA-4) and programmed-death 1 (PD-1) receptors. These proteins are members of the CD28-B7 family of cosignaling molecules that play important roles throughout all stages of T cell function. The PD-1 receptor (also known as CD279) is expressed on the surface of activated T cells. Its ligands, PD-L1 (B7-H1; CD274) and PD-L2 (B7-DC; CD273), are expressed on the surface of APCs such as dendritic cells or macrophages. PD-L1 is the predominant ligand, while PD-L2 has a much more restricted expression pattern. When the ligands bind to PD-1, an inhibitory signal is transmitted into the T cell, which reduces cytokine production and suppresses T-cell proliferation. Checkpoint inhibitors include, but are not limited to antibodies that block PD-1 (Nivolumab (BMS-936558 or MDX1106), CT-011, MK-3475), PD-L1 (MDX-1105 (BMS-936559), MPDL3280A, MSB0010718C), PD-L2 (rHIgM12B7), CTLA-4 (Ipilimumab (MDX-010), Tremelimumab (CP-675,206)), IDO, B7-H3 (MGA271), B7-H4, TIM3, LAG-3 (BMS-986016).

Human monoclonal antibodies to programmed death 1 (PD-1) and methods for treating cancer using anti-PD-1 antibodies alone or in combination with other immunotherapeutics are described in U.S. Pat. No. 8,008,449, which is incorporated by reference for these antibodies. Anti-PD-L1 antibodies and uses therefor are described in U.S. Pat. No. 8,552,154, which is incorporated by reference for these antibodies. Anticancer agent comprising anti-PD-1 antibody or anti-PD-L1 antibody are described in U.S. Pat. No. 8,617,546, which is incorporated by reference for these antibodies.

In some embodiments, the PDL1 inhibitor comprises an antibody that specifically binds PDL1, such as BMS-936559 (Bristol-Myers Squibb) or MPDL3280A (Roche). In some embodiments, the PD1 inhibitor comprises an antibody that specifically binds PD1, such as lambrolizumab (Merck), nivolumab (Bristol-Myers Squibb), or MED14736 (AstraZeneca). Human monoclonal antibodies to PD-1 and methods for treating cancer using anti-PD-1 antibodies alone or in combination with other immunotherapeutics are described in U.S. Pat. No. 8,008,449, which is incorporated by reference for these antibodies. Anti-PD-L1 antibodies and uses therefor are described in U.S. Pat. No. 8,552,154, which is incorporated by reference for these antibodies. Anticancer agent comprising anti-PD-1 antibody or anti-PD-L1 antibody are described in U.S. Pat. No. 8,617,546, which is incorporated by reference for these antibodies.

The disclosed CARs can be used in combination with other cancer immunotherapies. There are two distinct types of immunotherapy: passive immunotherapy uses components of the immune system to direct targeted cytotoxic activity against cancer cells, without necessarily initiating an immune response in the patient, while active immunotherapy actively triggers an endogenous immune response. Passive strategies include the use of the monoclonal antibodies (mAbs) produced by B cells in response to a specific antigen. The development of hybridoma technology in the 1970s and the identification of tumor-specific antigens permitted the pharmaceutical development of mAbs that could specifically target tumor cells for destruction by the immune system. Thus far, mAbs have been the biggest success story for immunotherapy; the top three best-selling anticancer drugs in 2012 were mAbs. Among them is rituximab (Rituxan, Genentech), which binds to the CD20 protein that is highly expressed on the surface of B cell malignancies such as non-Hodgkin's lymphoma (NHL). Rituximab is approved by the FDA for the treatment of NHL and chronic lymphocytic leukemia (CLL) in combination with chemotherapy. Another important mAb is trastuzumab (Herceptin; Genentech), which revolutionized the treatment of HER2 (human epidermal growth factor receptor 2)-positive breast cancer by targeting the expression of HER2.

Generating optimal “killer” CD8 T cell responses also requires T cell receptor activation plus co-stimulation, which can be provided through ligation of tumor necrosis factor receptor family members, including OX40 (CD134) and 4-1BB (CD137). OX40 is of particular interest as treatment with an activating (agonist) anti-OX40 mAb augments T cell differentiation and cytolytic function leading to enhanced anti-tumor immunity against a variety of tumors.

In some embodiments, such an additional therapeutic agent may be selected from an antimetabolite, such as methotrexate, 6-mercaptopurine, 6-thioguanine, cytarabine, fludarabine, 5-fluorouracil, decarbazine, hydroxyurea, asparaginase, gemcitabine or cladribine.

In some embodiments, such an additional therapeutic agent may be selected from an alkylating agent, such as mechlorethamine, thioepa, chlorambucil, melphalan, carmustine (BSNU), lomustine (CCNU), cyclophosphamide, busulfan, dibromomannitol, streptozotocin, dacarbazine (DTIC), procarbazine, mitomycin C, cisplatin and other platinum derivatives, such as carboplatin .

In some embodiments, such an additional therapeutic agent may be selected from an anti-mitotic agent, such as taxanes, for instance docetaxel, and paclitaxel, and vinca alkaloids, for instance vindesine, vincristine, vinblastine, and vinorelbine.

In some embodiments, such an additional therapeutic agent may be selected from a topoisomerase inhibitor, such as topotecan or irinotecan, or a cytostatic drug, such as etoposide and teniposide.

In some embodiments, such an additional therapeutic agent may be selected from a growth factor inhibitor, such as an inhibitor of ErbBl (EGFR) (such as an EGFR antibody, e.g. zalutumumab, cetuximab, panitumumab or nimotuzumab or other EGFR inhibitors, such as gefitinib or erlotinib), another inhibitor of ErbB2 (HER2/neu) (such as a HER2 antibody, e.g. trastuzumab, trastuzumab-DM I or pertuzumab) or an inhibitor of both EGFR and HER2, such as lapatinib).

In some embodiments, such an additional therapeutic agent may be selected from a tyrosine kinase inhibitor, such as imatinib (Glivec, Gleevec STI571) or lapatinib.

Therefore, in some embodiments, a disclosed antibody is used in combination with ofatumumab, zanolimumab, daratumumab, ranibizumab, nimotuzumab, panitumumab, hu806, daclizumab (Zenapax), basiliximab (Simulect), infliximab (Remicade), adalimumab (Humira), natalizumab (Tysabri), omalizumab (Xolair), efalizumab (Raptiva), and/or rituximab.

In some embodiments, a therapeutic agent for use in combination with a CARs for treating the disorders as described above may be an anti-cancer cytokine, chemokine, or combination thereof. Examples of suitable cytokines and growth factors include IFNy, IL-2, IL-4, IL-6, IL-7, IL-10, IL-12, IL-13, IL-15, IL-18, IL-21, IL-23, IL-24, IL-27, IL-28a, IL-28b, IL-29, KGF, IFNa (e.g., INFa2b), IFN , GM-CSF, CD40L, Flt3 ligand, stem cell factor, ancestim, and TNFa. Suitable chemokines may include Glu-Leu-Arg (ELR)-negative chemokines such as IP-10, MCP-3, MIG, and SDF-Ia from the human CXC and C-C chemokine families. Suitable cytokines include cytokine derivatives, cytokine variants, cytokine fragments, and cytokine fusion proteins.

In some embodiments, a therapeutic agent for use in combination with a CARs for treating the disorders as described above may be a cell cycle control/apoptosis regulator (or “regulating agent”). A cell cycle control/apoptosis regulator may include molecules that target and modulate cell cycle control/apoptosis regulators such as (i) cdc-25 (such as NSC 663284), (ii) cyclin-dependent kinases that overstimulate the cell cycle (such as flavopiridol (L868275, HMR1275), 7-hydroxystaurosporine (UCN-01, KW-2401), and roscovitine (R-roscovitine, CYC202)), and (iii) telomerase modulators (such as BIBR1532, SOT-095, GRN163 and compositions described in for instance U.S. Pat. Nos. 6,440,735 and 6,713,055). Non-limiting examples of molecules that interfere with apoptotic pathways include TNF-related apoptosis-inducing ligand (TRAIL)/apoptosis-2 ligand (Apo-2L), antibodies that activate TRAIL receptors, IFNs, and anti-sense Bcl-2.

In some embodiments, a therapeutic agent for use in combination with a CARs for treating the disorders as described above may be a hormonal regulating agent, such as agents useful for anti-androgen and anti-estrogen therapy. Examples of such hormonal regulating agents are tamoxifen, idoxifene, fulvestrant, droloxifene, toremifene, raloxifene, diethylstilbestrol, ethinyl estradiol/estinyl, an antiandrogene (such as flutaminde/eulexin), a progestin (such as such as hydroxyprogesterone caproate, medroxy- progesterone/provera, megestrol acepate/megace), an adrenocorticosteroid (such as hydrocortisone, prednisone), luteinizing hormone-releasing hormone (and analogs thereof and other LHRH agonists such as buserelin and goserelin), an aromatase inhibitor (such as anastrazole/arimidex, aminoglutethimide/cytraden, exemestane) or a hormone inhibitor (such as octreotide/sandostatin).

In some embodiments, a therapeutic agent for use in combination with an CARs for treating the disorders as described above may be an anti-cancer nucleic acid or an anti-cancer inhibitory RNA molecule.

Combined administration, as described above, may be simultaneous, separate, or sequential. For simultaneous administration the agents may be administered as one composition or as separate compositions, as appropriate.

In some embodiments, the disclosed CARs is administered in combination with radiotherapy. Radiotherapy may comprise radiation or associated administration of radiopharmaceuticals to a patient is provided. The source of radiation may be either external or internal to the patient being treated (radiation treatment may, for example, be in the form of external beam radiation therapy (EBRT) or brachytherapy (BT)). Radioactive elements that may be used in practicing such methods include, e.g., radium, cesium-137, iridium-192, americium-241, gold-198, cobalt-57, copper-67, technetium-99, iodide-123, iodide-131, and indium-111.

In some embodiments, the disclosed CARs is administered in combination with surgery.

CAR-T cells may be designed in several ways that enhance tumor cytotoxicity and specificity, evade tumor immunosuppression, avoid host rejection, and prolong their therapeutic half-life. TRUCK (T-cells Redirected for Universal Cytokine Killing) T cells for example, possess a CAR but are also engineered to release cytokines such as IL-12 that promote tumor killing. Because these cells are designed to release a molecular payload upon activation of the CAR once localized to the tumor environment, these CAR-T cells are sometimes also referred to as ‘armored CARs’. Several cytokines as cancer therapies are being investigated both pre-clinically and clinically, and may also prove useful when similarly incorporated into a TRUCK form of CAR-T therapy. Among these include IL-2, IL-3. IL-4, IL-5, IL-6, IL-7, IL-10, IL-12, IL-13, IL-15, IL-18, M-CSF, GM-CSF, IFN-α, IFN-γ, TNF-α, TRAIL, FLT3 ligand, Lymphotactin, and TGF-β (Dranoff 2004). “Self-driving” or “homing” CAR-T cells are engineered to express a chemokine receptor in addition to their CAR. As certain chemokines can be upregulated in tumors, incorporation of a chemokine receptor aids in tumor trafficking to and infiltration by the adoptive T-cell, thereby enhancing both specificity and functionality of the CAR-T (Moon 2011). Universal CAR-T cells also possess a CAR, but are engineered such that they do not express endogenous TCR (T-cell receptor) or MHC (major histocompatibility complex) proteins. Removal of these two proteins from the signaling repertoire of the adoptive T-cell therapy prevents graft-versus-host-disease and rejection, respectively. Armored CAR-T cells are additionally so named for their ability to evade tumor immunosuppression and tumor-induced CAR-T hypofunction. These particular CAR-Ts possess a CAR, and may be engineered to not express checkpoint inhibitors. Alternatively, these CAR-Ts can be co-administered with a monoclonal antibody (mAb) that blocks checkpoint signaling. Administration of an anti-PDL1 antibody significantly restored the killing ability of CAR TILs (tumor infiltrating lymphocytes). While PD1-PDL1 and CTLA-4-CD80/CD86 signaling pathways have been investigated, it is possible to target other immune checkpoint signaling molecules in the design of an armored CAR-T including LAG-3, Tim-3, IDO-1, 2B4, and KIR. Other intracellular inhibitors of TILs include phosphatases (SHP1), ubiquitin-ligases (i.e., cbl-b), and kinases (i.e., diacylglycerol kinase). Armored CAR-Ts may also be engineered to express proteins or receptors that protect them against or make them resistant to the effects of tumor-secreted cytokines. For example, CTLs (cytotoxic T lymphocytes) transduced with the double negative form of the TGF-β receptor are resistant to the immunosuppression by lymphoma secreted TGF-β. These transduced cells showed notably increased antitumor activity in vivo when compared to their control counterparts.

Tandem and dual CAR-T cells are unique in that they possess two distinct antigen binding domains. A tandem CAR contains two sequential antigen binding domains facing the extracellular environment connected to the intracellular costimulatory and stimulatory domains. A dual CAR is engineered such that one extracellular antigen binding domain is connected to the intracellular costimulatory domain and a second, distinct extracellular antigen binding domain is connected to the intracellular stimulatory domain. Because the stimulatory and costimulatory domains are split between two separate antigen binding domains, dual CARs are also referred to as “split CARs”. In both tandem and dual CAR designs, binding of both antigen binding domains is necessary to allow signaling of the CAR circuit in the T-cell. Because these two CAR designs have binding affinities for different, distinct antigens, they are also referred to as “bi-specific” CARs.

One primary concern with CAR-T cells as a form of “living therapeutic” is their manipulability in vivo and their potential immune-stimulating side effects. To better control CAR-T therapy and prevent against unwanted side effects, a variety of features have been engineered including off-switches, safety mechanisms, and conditional control mechanisms. Both self-destruct and marked/tagged CAR-T cells for example, are engineered to have an “off-switch” that promotes clearance of the CAR-expressing T-cell. A self-destruct CAR-T contains a CAR, but is also engineered to express a pro-apoptotic suicide gene or “elimination gene” inducible upon administration of an exogenous molecule. A variety of suicide genes may be employed for this purpose, including HSV-TK (herpes simplex virus thymidine kinase), Fas, iCasp9 (inducible caspase 9), CD20, MYC tag, and truncated EGFR (endothelial growth factor receptor). HSK for example, will convert the prodrug ganciclovir (GCV) into GCV-triphosphate that incorporates itself into replicating DNA, ultimately leading to cell death. iCasp9 is a chimeric protein containing components of FK506-binding protein that binds the small molecule AP1903, leading to caspase 9 dimerization and apoptosis. A marked/tagged CAR-T cell however, is one that possesses a CAR but also is engineered to express a selection marker. Administration of a mAb against this selection marker will promote clearance of the CAR-T cell. Truncated EGFR is one such targetable antigen by the anti-EGFR mAb, and administration of cetuximab works to promotes elimination of the CAR-T cell. CARs created to have these features are also referred to as sCARs for ‘switchable CARs’, and RCARs for ‘regulatable CARs’. A “safety CAR”, also known as an “inhibitory CAR” (iCAR), is engineered to express two antigen binding domains. One of these extracellular domains is directed against a tumor related antigen and bound to an intracellular costimulatory and stimulatory domain. The second extracellular antigen binding domain however is specific for normal tissue and bound to an intracellular checkpoint domain such as CTLA4, PD1, or CD45. Incorporation of multiple intracellular inhibitory domains to the iCAR is also possible. Some inhibitory molecules that may provide these inhibitory domains include B7-H1, B7-1, CD160, PIH, 2B4, CEACAM (CEACAM-1. CEACAM-3, and/or CEACAM-5), LAG-3, TIGIT, BTLA, LAIR1, and TGFβ-R. In the presence of normal tissue, stimulation of this second antigen binding domain will work to inhibit the CAR. It should be noted that due to this dual antigen specificity, iCARs are also a form of bi-specific CAR-T cells. The safety CAR-T engineering enhances specificity of the CAR-T cell for tumor tissue, and is advantageous in situations where certain normal tissues may express very low levels of a tumor associated antigen that would lead to off target effects with a standard CAR (Morgan 2010). A conditional CAR-T cell expresses an extracellular antigen binding domain connected to an intracellular costimulatory domain and a separate, intracellular costimulator. The costimulatory and stimulatory domain sequences are engineered in such a way that upon administration of an exogenous molecule the resultant proteins will come together intracellularly to complete the CAR circuit. In this way, CAR-T activation can be modulated, and possibly even ‘fine-tuned’ or personalized to a specific patient. Similar to a dual CAR design, the stimulatory and costimulatory domains are physically separated when inactive in the conditional CAR; for this reason these too are also referred to as a “split CAR”.

In some embodiments, two or more of these engineered features may be combined to create an enhanced, multifunctional CAR-T. For example, it is possible to create a CAR-T cell with either dual- or conditional- CAR design that also releases cytokines like a TRUCK. In some embodiments, a dual-conditional CAR-T cell could be made such that it expresses two CARs with two separate antigen binding domains against two distinct cancer antigens, each bound to their respective costimulatory domains. The costimulatory domain would only become functional with the stimulatory domain after the activating molecule is administered. For this CAR-T cell to be effective the cancer must express both cancer antigens and the activating molecule must be administered to the patient; this design thereby incorporating features of both dual and conditional CAR-T cells.

Typically, CAR-T cells are created using α-β T cells, however γ-δ T cells may also be used. In some embodiments, the described CAR constructs, domains, and engineered features used to generate CAR-T cells could similarly be employed in the generation of other types of CAR-expressing immune cells including NK (natural killer) cells, B cells, mast cells, myeloid-derived phagocytes, and NKT cells. Alternatively, a CAR-expressing cell may be created to have properties of both T-cell and NK cells. In an additional embodiment, the transduced with CARs may be autologous or allogeneic.

Several different methods for CAR expression may be used including retroviral transduction (including γ-retroviral), lentiviral transduction, transposon/transposases (Sleeping Beauty and PiggyBac systems), and messenger RNA transfer-mediated gene expression. Gene editing (gene insertion or gene deletion/disruption) has become of increasing importance with respect to the possibility for engineering CAR-T cells as well. CRISPR-Cas9, ZFN (zinc finger nuclease), and TALEN (transcription activator like effector nuclease) systems are three potential methods through which CAR-T cells may be generated.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: PGC-1α Mutant Expression During Transduction and Expansion Produces More Central and Effector Memory CAR T Cells and Reduces Short Lived Effector Cells

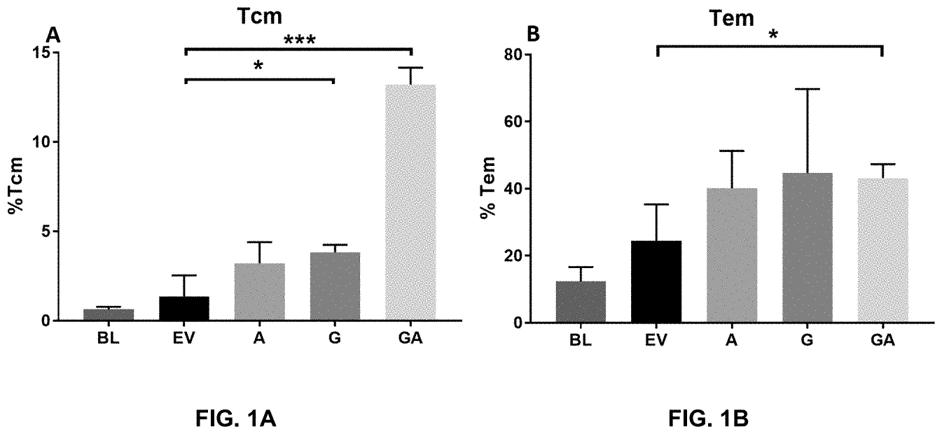

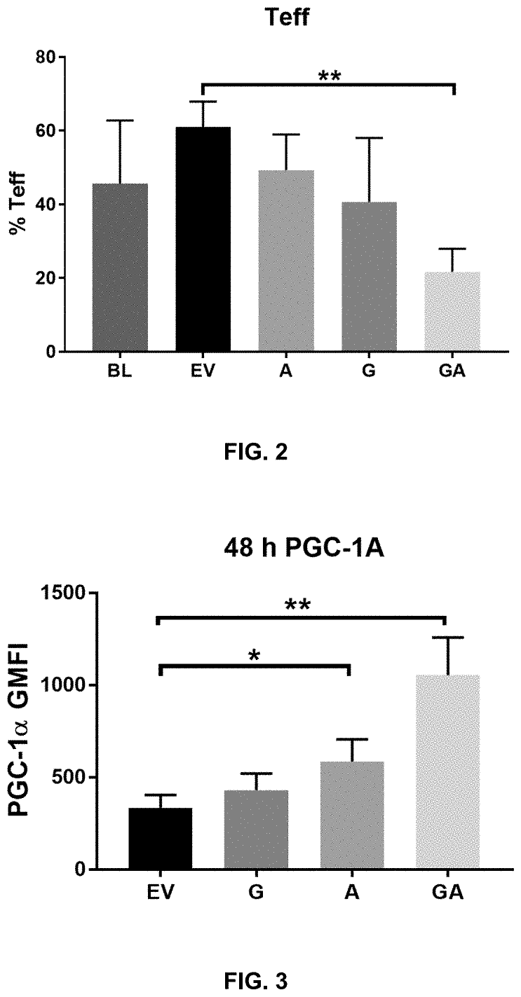

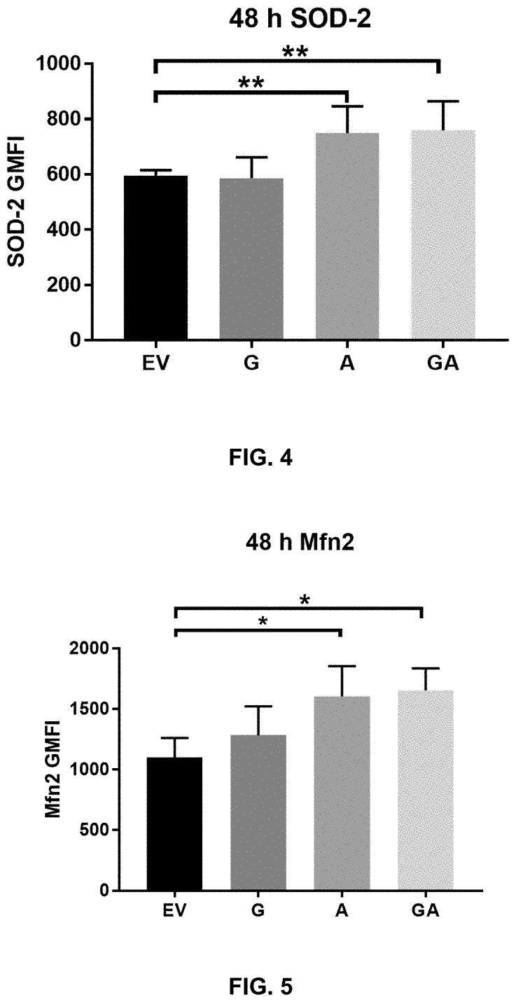

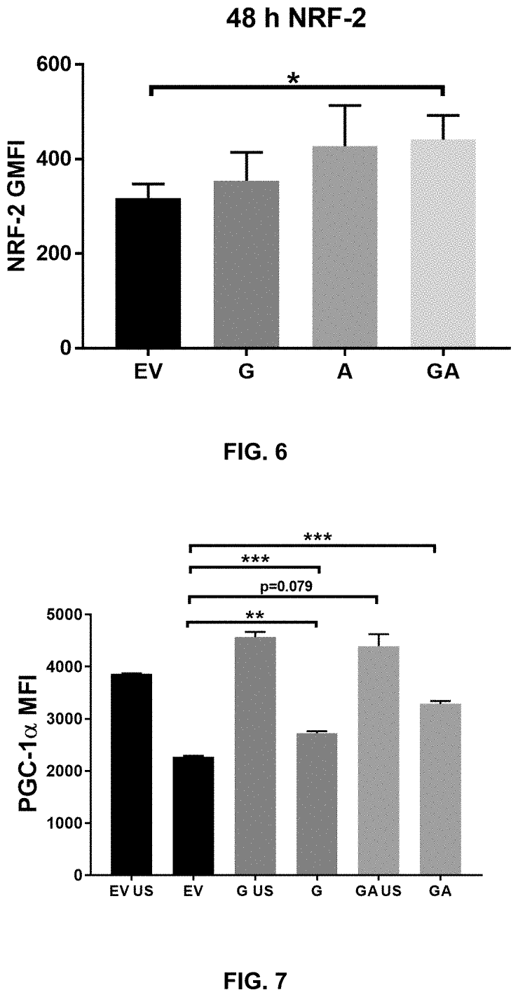

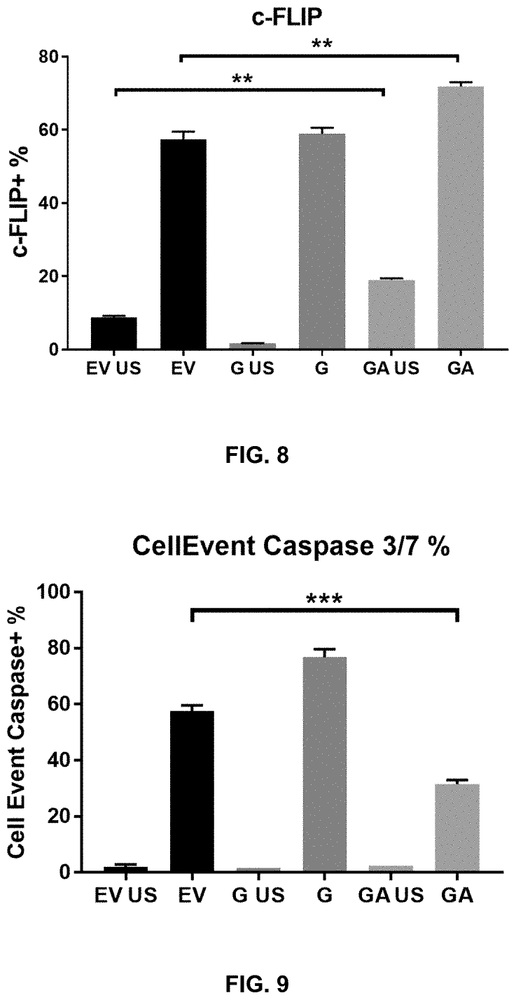

Co-transduction of CD8 T cells with Chimeric Antigen Receptor (CAR) encoded retrovirus and mutant PGC-1α encoded retro virus increases the percentage of central memory CAR cells (Tcm) and effector memory CAR T cells (Tem) present after transduction (FIG. 1). Co-transduction of CD8 T cells with Chimeric Antigen Receptor (CAR) encoded retrovirus and mutant PGC-1α encoded retrovirus decreases the percentage of effector CAR T cells (Teff) present after transduction (FIG. 2). Mutant PGC-1A is resistant to resistant respective deactivation and proteasomal degradation by Akt and GSK3β after activation by CD-19 (FIG. 3). Mutant PGC-1A expression increases defense against oxidative stress within mitochondria after activation relative to control CAR T (FIG. 4). Mutant PGC-1A expression increases mitochondrial fusion protein to create fused mitochondrial morphology associated with improved metabolic fitness and memory phenoytpes (FIG. 5).

### Example 2: PGC-1α Mutant Expression During CAR T Activation Increases Expression of Transcription Factors Associated with Metabolic Fitness and Maintenance of Memory Phenotypes

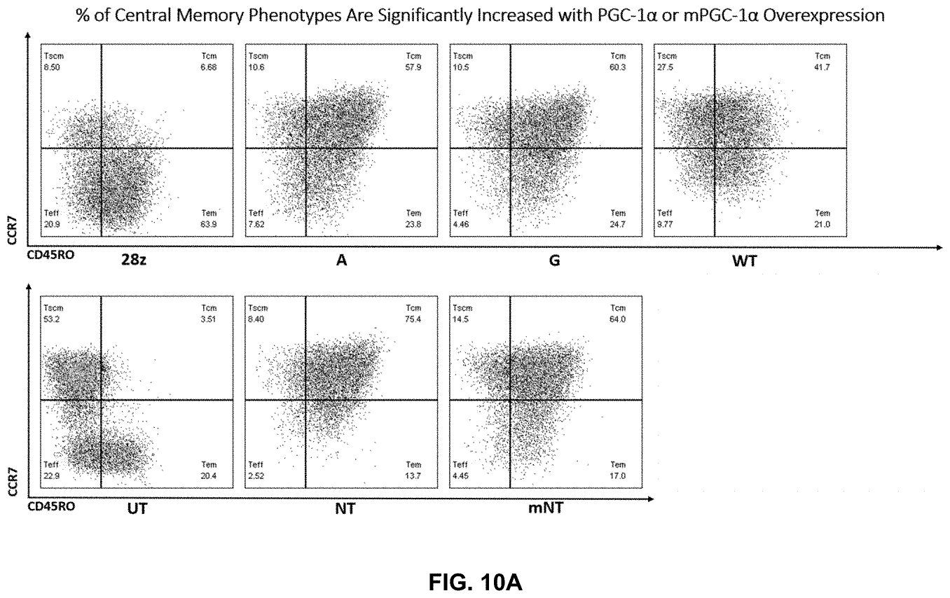

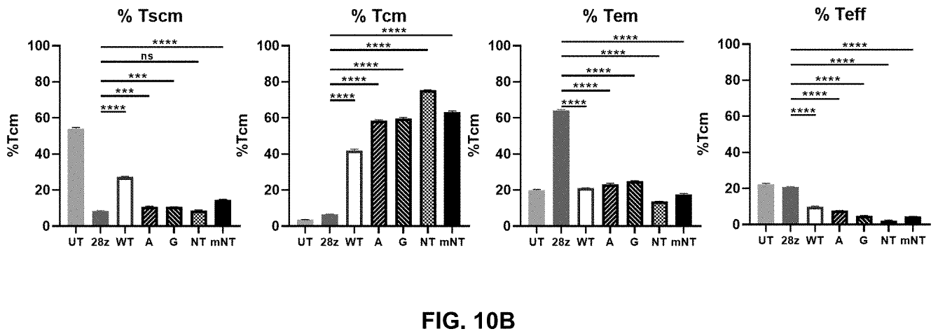

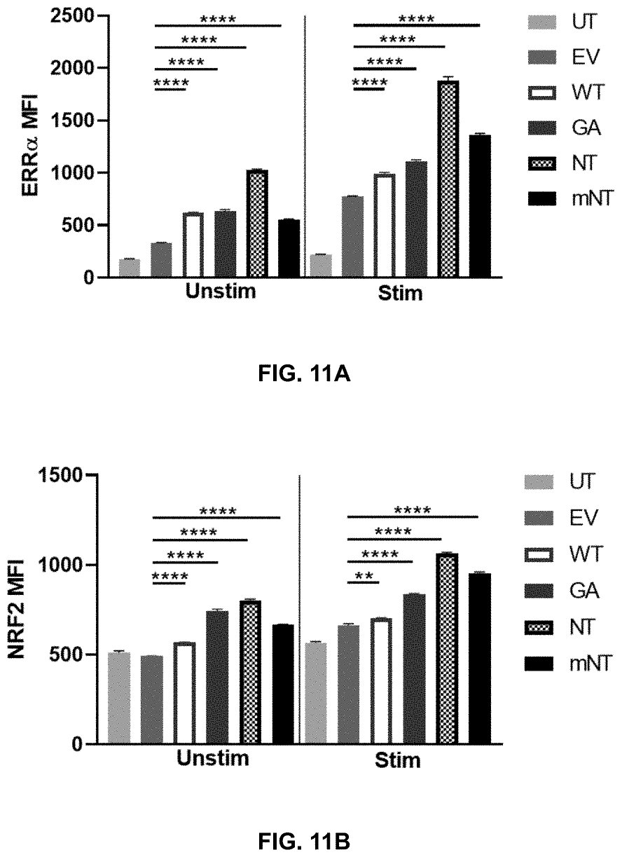

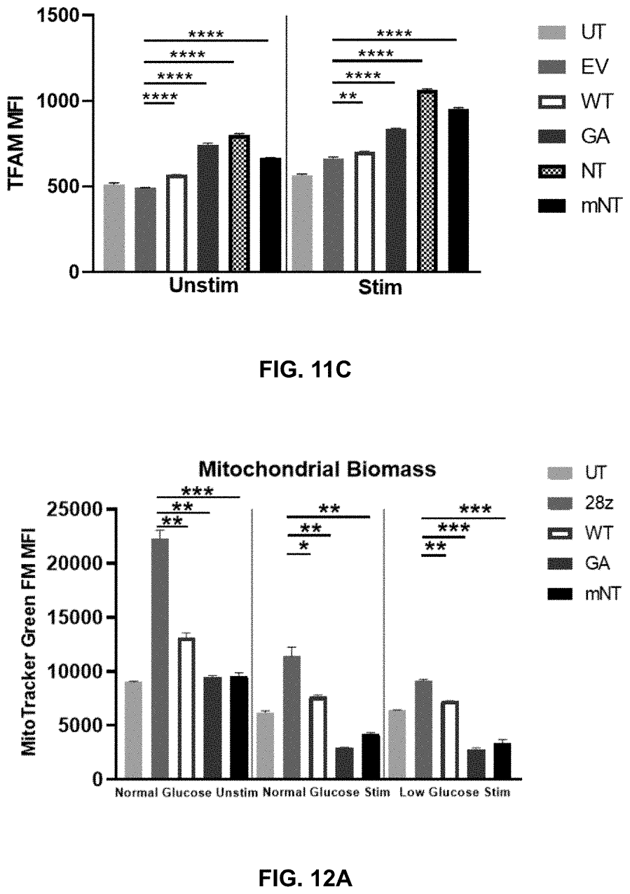

Mutant PGC-1A expression upregulates Nuclear Respiratory Factor 2 (NRF-2), a transcription the drives the expression of electron transport chain complex subunits to increase efficiency of oxidative metabolism and fitness (FIG. 6). Mutant PGC-1A expression is increased at rest and after short 8 hour activation (FIG. 7). Anti-apoptotic protein c-FLIP is increased in Mutant PGC-1A expressing CAR T cells which may prevent activation induced cell death (FIG. 8). The percentage of apoptotic cells is decreased in Mutant PGC-1A expressing CAR T cells (FIG. 9).

### Example 3: Metabolically Flexible CAR T Cells (mfCAR-T), with Constitutive Expression of PGC-1α Resistant to Post Translational Modifications, Exhibit Superior Survival and Function In Vitro

Background

Remarkable durable responses are seen with chimeric antigen receptor (CAR) T cell therapy in B cell lymphoma, however the majority of patients relapse (Locke et al. Lancet Oncol. 2019). Improvements enabling CAR T cells (CAR-T) to circumvent mechanisms of resistance may increase efficacy. Hypoxia, nutrient deprivation and acidosis, all common in the tumor microenvironment (TME), impair metabolic function necessary for CAR-T to kill tumor (Chang et al. Cell 2015). The metabolic response gene peroxisome proliferator-activated receptor gamma coactivator 1 alpha (PGC-1α) co-activates genes that upregulate mitochondrial and glycolytic machinery for ATP synthesis from myriad carbon sources. Post translational modifications (PTM) fine tune PGC-1α activity to meet energy demands (Luo et al. IJC 2019). It was hypothesized that CAR-T co-expressing full-length PGC-1α or the truncated (ie. short) NT-PGC-1α isoform, with mutations that prevent suppressive PTMs, would confer metabolic flexibility to improve function under TME conditions.

Methods

Four PGC-1α encoded retroviral vectors were constructed with an IRES and DsRed fluorescent protein: full-length wild type (WT); full-length mutant (GA); wild type short isoform (NT); and mutant short isoform (mNT). GA contained T295A and S571A mutations to abrogate GSK3β and Akt mediated PTMs. mNT sequence contained K to A mutations at K78/K145/K184/K254 to prevent acetylation by GCN5, and L to A mutations of the nuclear export sequence corresponding to L29/L33/L36/L38. Human CD8 T cells were activated with αCD3/αCD28 beads+100 IU IL-2/mL, and transduced at 48 hr. to express FMC63-CD28/CD3z CAR and non-functional truncated CD34. Cells were co-transduced with WT, or in the case of metabolically flexible CAR T cells (mfCAR-T) with a mutant and/or short isoform PGC-1α vector. After 7 days of expansion CD34+DsRed+ cells were isolated by FACS. In vitro experiments were performed within 2 weeks to characterize mitochondrial dynamics/oxidative stress (flow cytometry), cytokine secretion (ELISA), and real-time cytotoxicity (xCelligence). The effect of glucose restriction was evaluated in normal (10 mM) and low glucose (0.01 mM) medium. A Mitochondrial stress test (Seahorse) was performed 30 days after FACS. CAR-T (WT and control w/o co-transduction) and mfCAR-T were stimulated with CD19+ K562 or 3T3 cells.

Results

Representative PGC-1α metabolic fitness target genes (ERRα, TFAM, and NRF2) were increased in mfCAR T cells (p≤0.001). mfCAR-T exhibited decreased mitochondrial biomass (p≤0.01) and mitochondrial membrane potential (MMP) (p≤0.01) in both glucose conditions. However, MMP:mitochondrial biomass and autophagy were greater (p≤0.01, p≤0.001), suggesting accelerated mitochondrial quality control (MQC). Oxidative stress was generally decreased (p≤0.01) in mfCAR-T, accompanied by reduced apoptosis. All mfCAR and control CAR T cells cytolysed 100% of targets at a 1:1 ratio but differed in cytolytic rate. Relative to CAR only, WT CAR-T and GA mfCAR-T killed 1.6 and 1.9 times faster, while shorter isoforms required 1.9 times longer to lyse all targets. IFNγ and IL-2 secretion by GA-mfCAR-T was increased above control CAR-T and other mfCAR T cells (p≤0.01), while others were similar. At 30 days both WT-CAR-T and all 3 mfCAR-T had increased spare respiratory capacity (SRC) compared to control CAR-T (p≤0.05); however ATP production and OCR/ECAR was increased (p≤0.001, p≤0.0.05) in mfCAR-T above control CAR-T and WT-CAR-T.

Conclusion

Enforced expression of mutant or truncated PGC-1α in CAR-T enhanced mitochondrial quality control with commensurate function. mfCAR-T cells exhibited equivalent cytotoxicity in vitro, improved survival, and a metabolism less reliant on glucose. Stark differences in SRC, OCR/ECAR, and mitochondrial ATP production between WT and mfCAR-T suggest signaling pathways in CAR T cells may target PTM mediated suppression of PGC-1α and lead to metabolic exhaustion in the TME. mfCAR-T are a promising new strategy to improve the function of CAR-T cells in the TME. Further in vitro and in vivo experiments are needed to validate the approach.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A chimeric antigen receptor T (CAR-T) cell for use in adoptive cell therapy, comprising T cells engineered to express a chimeric antigen receptor (CAR) polypeptide and
(a) a mutant PGC-1α having an amino acid mutation at T295, S569, S573, S577, S579, S581, S599, S616, S624, S629, S636, or any combination thereof;
(b) a wildtype NT-PGC-1α polypeptide;
(c) a mutant NT-PGC-1α polypeptide having an amino acid mutation at L29, L33, L36, L38, K78, L92, L96, L99, V101, K145, V183, K184, T185, E186, S195, S242, K254, T257, T263, S266, L269, or any combination thereof; or
(d) any combination of (a), (b), or (c).

2. The CAR-T cell of claim 1, wherein the mutant PGC-1α further comprises an amino acid mutation at S571.

3. The CAR-T cell of claim 1, wherein the mutant PGC-1α comprises the amino acid sequence SEQ ID NO:3, SEQ ID NO:4, or SEQ ID NO:5.

4. The CAR-T cell of claim 1, wherein the NT-PGC-1α comprises the amino acid sequence SEQ ID NO:2, or a conservative variant thereof having at least 90% sequence identity to SEQ ID NO:2.

5. The CAR-T cell of claim 1, wherein the mutant NT-PGC-1α comprises the amino acid sequence SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, or SEQ ID NO:15.

6. A method for enhancing metabolic fitness of a CAR-T cell, comprising transducing the CAR-T cell with a vector encoding
(e) a mutant PGC-1α having an amino acid mutation at T295, S569, S573, S577, S579, S581, S599, S616, S624, S629, S636, or any combination thereof;
(f) a wildtype NT-PGC-1α polypeptide;
(g) a mutant NT-PGC-1α polypeptide having an amino acid mutation at L29, L33, L36, L38, K78, L92, L96, L99, V101, K145, V183, K184, T185, E186, S195, S242, K254, T257, T263, S266, L269, or any combination thereof; or
(h) any combination of (a), (b), or (c).

7. The method of claim 6, wherein the mutant PGC-1α further comprises an amino acid mutation at S571.

8. The method of claim 6 or 7, wherein the mutant PGC-1α comprises the amino acid sequence SEQ ID NO:3, SEQ ID NO:4, or SEQ ID NO:5.

9. The method of claim 6, wherein the NT-PGC-1α comprises the amino acid sequence SEQ ID NO:2, or a conservative variant thereof having at least 90% sequence identity to SEQ ID NO:2.

10. The method of claim 6, wherein the mutant NT-PGC-1α comprises the amino acid sequence SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, or SEQ ID NO:15.

11. A method for producing CAR-T cells, comprising
(i) isolating PBMCs from a donor,
(j) isolating T cells from the PBMCs,
(k) stimulating the T cells with CD3/CD28 beads,
(l) transducing the activated T cells with a viral vector encoding a CAR polypeptide,
(m) transducing the activated T cells with a viral vector encoding:
(1) a mutant PGC-1α having an amino acid mutation at T295, S569, S573, S577, S579, S581, S599, S616, S624, S629, S636, or any combination thereof;
(2) a wildtype NT-PGC-1α polypeptide;
(3) a mutant NT-PGC-1α polypeptide having an amino acid mutation at L29, L33, L36, L38, K78, L92, L96, L99, V101, K145, V183, K184, T185, E186, S195, S242, K254, T257, T263, S266, L269, or any combination thereof; or
(4) any combination of (i), (ii), or (iii); and
(n) expanding the CAR-T cells.

12. The method of claim 11, wherein the mutant PGC-1α further comprises an amino acid mutation at S571.

13. The method of claim 11, wherein the mutant PGC-1α comprises the amino acid sequence SEQ ID NO:3, SEQ ID NO:4, or SEQ ID NO:5.

14. The method of claim 11, wherein the NT-PGC-1α comprises the amino acid sequence SEQ ID NO:2, or a conservative variant thereof having at least 90% sequence identity to SEQ ID NO:2.

15. The method of claim 11, wherein the mutant NT-PGC-1α comprises the amino acid sequence SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, or SEQ ID NO:15.

16. A vector comprising a nucleic acid sequence encoding both a CAR polypeptide and a mutant PGC-1α, a wildtype NT-PGC-1α, or a mutant NT-PGC-1α polypeptide.

17-23. (canceled)


SEQUENCE LISTING (15 sequences)

SEQ ID NO: 1 - protein, 798 aa, Homo sapiens
MAWDMCNQDSESVWSDIECAALVGEDQPLCPDLPELDLSELDVNDLDTDSFLGGLKWCSDQSEIISNQYNNEPSNIFEKIDEENEANLLAVLTETLDSLPVDEDGLPSFDALTDGDVTTDNEASPSSMPDGTPPPQEAEEPSLLKKLLLAPANTQLSYNECSGLSTQNHANHNHRIRTNPAIVKTENSWSNKAKSICQQQKPQRRPCSELLKYLTTNDDPPHTKPTENRNSSRDKCTSKKKSHTQSQSQHLQAKPTTLSLPLTPESPNDPKGSPFENKTIERTLSVELSGTAGLTPPTTPPHKANQDNPFRASPKLKSSCKTVVPPPSKKPRYSESSGTQGNNSTKKGPEQSELYAQLSKSSVLTGGHEERKTKRPSLRLFGDHDYCQSINSKTEILINISQELQDSRQLENKDVSSDWQGQICSSTDSDQCYLRETLEASKQVSPCSTRKQLQDQEIRAELNKHFGHPSQAVFDDEADKTGELRDSDFSNEQFSKLPMFINSGLAMDGLFDDSEDESDKLSYPWDGTQSYSLFNVSPSCSSFNSPCRDSVSPPKSLFSQRPQRMRSRSRSFSRHRSCSRSPYSRSRSRSPGSRSSSRSCYYYESSHYRHRTHRNSPLYVRSRSRSPYSRRPRYDSYEEYQHERLKREEYRREYEKRESERAKQRERQRQKAIEERRVIYVGKIRPDTTRTELRDRFEVFGEIEECTVNLRDDGDSYGFITYRYTCDAFAALENGYTLRRSNETDFELYFCGRKQFFKSNYADLDSNSDDFDPASTKSKYDSLDFDSLLKEAQRSLRR

SEQ ID NO: 2 - protein, 271 aa, Homo sapiens
MAWDMCNQDSESVWSDIECAALVGEDQPLCPDLPELDLSELDVNDLDTDSFLGGLKWCSDQSEIISNQYNNEPSNIFEKIDEENEANLLAVLTETLDSLPVDEDGLPSFDALTDGDVTTDNEASPSSMPDGTPPPQEAEEPSLLKKLLLAPANTQLSYNECSGLSTQNHANHNHRIRTNPAIVKTENSWSNKAKSICQQQKPQRRPCSELLKYLTTNDDPPHTKPTENRNSSRDKCTSKKKSHTQSQSQHLQAKPTTLSLPLTPESPNLFL

SEQ ID NO: 3 - protein, 798 aa, Artificial Sequence
  Synthetic Construct
MAWDMCNQDSESVWSDIECAALVGEDQPLCPDLPELDLSELDVNDLDTDSFLGGLKWCSDQSEIISNQYNNEPSNIFEKIDEENEANLLAVLTETLDSLPVDEDGLPSFDALTDGDVTTDNEASPSSMPDGTPPPQEAEEPSLLKKLLLAPANTQLSYNECSGLSTQNHANHNHRIRTNPAIVKTENSWSNKAKSICQQQKPQRRPCSELLKYLTTNDDPPHTKPTENRNSSRDKCTSKKKSHTQSQSQHLQAKPTTLSLPLTPESPNDPKGSPFENKTIERTLSVELSGTAGLAPPTTPPHKANQDNPFRASPKLKSSCKTVVPPPSKKPRYSESSGTQGNNSTKKGPEQSELYAQLSKSSVLTGGHEERKTKRPSLRLFGDHDYCQSINSKTEILINISQELQDSRQLENKDVSSDWQGQICSSTDSDQCYLRETLEASKQVSPCSTRKQLQDQEIRAELNKHFGHPSQAVFDDEADKTGELRDSDFSNEQFSKLPMFINSGLAMDGLFDDSEDESDKLSYPWDGTQSYSLFNVSPSCSSFNSPCRDSVSPPKSLFSQRPQRMRSRSRSFSRHRSCSRSPYSRSRSRSPGSRSSSRSCYYYESSHYRHRTHRNSPLYVRSRSRSPYSRRPRYDSYEEYQHERLKREEYRREYEKRESERAKQRERQRQKAIEERRVIYVGKIRPDTTRTELRDRFEVFGEIEECTVNLRDDGDSYGFITYRYTCDAFAALENGYTLRRSNETDFELYFCGRKQFFKSNYADLDSNSDDFDPASTKSKYDSLDFDSLLKEAQRSLRR

SEQ ID NO: 4 - protein, 798 aa, Artificial Sequence
  Synthetic Construct
MAWDMCNQDSESVWSDIECAALVGEDQPLCPDLPELDLSELDVNDLDTDSFLGGLKWCSDQSEIISNQYNNEPSNIFEKIDEENEANLLAVLTETLDSLPVDEDGLPSFDALTDGDVTTDNEASPSSMPDGTPPPQEAEEPSLLKKLLLAPANTQLSYNECSGLSTQNHANHNHRIRTNPAIVKTENSWSNKAKSICQQQKPQRRPCSELLKYLTTNDDPPHTKPTENRNSSRDKCTSKKKSHTQSQSQHLQAKPTTLSLPLTPESPNDPKGSPFENKTIERTLSVELSGTAGLAPPTTPPHKANQDNPFRASPKLKSSCKTVVPPPSKKPRYSESSGTQGNNSTKKGPEQSELYAQLSKSSVLTGGHEERKTKRPSLRLFGDHDYCQSINSKTEILINISQELQDSRQLENKDVSSDWQGQICSSTDSDQCYLRETLEASKQVSPCSTRKQLQDQEIRAELNKHFGHPSQAVFDDEADKTGELRDSDFSNEQFSKLPMFINSGLAMDGLFDDSEDESDKLSYPWDGTQSYSLFNVSPSCSSFNSPCRDSVSPPKSLFSQRPQRMRSRSRAFSRHRSCSRSPYSRSRSRSPGSRSSSRSCYYYESSHYRHRTHRNSPLYVRSRSRSPYSRRPRYDSYEEYQHERLKREEYRREYEKRESERAKQRERQRQKAIEERRVIYVGKIRPDTTRTELRDRFEVFGEIEECTVNLRDDGDSYGFITYRYTCDAFAALENGYTLRRSNETDFELYFCGRKQFFKSNYADLDSNSDDFDPASTKSKYDSLDFDSLLKEAQRSLRR

SEQ ID NO: 5 - protein, 798 aa, Artificial Sequence
  Synthetic Construct
MAWDMCNQDSESVWSDIECAALVGEDQPLCPDLPELDLSELDVNDLDTDSFLGGLKWCSDQSEIISNQYNNEPSNIFEKIDEENEANLLAVLTETLDSLPVDEDGLPSFDALTDGDVTTDNEASPSSMPDGTPPPQEAEEPSLLKKLLLAPANTQLSYNECSGLSTQNHANHNHRIRTNPAIVKTENSWSNKAKSICQQQKPQRRPCSELLKYLTTNDDPPHTKPTENRNSSRDKCTSKKKSHTQSQSQHLQAKPTTLSLPLTPESPNDPKGSPFENKTIERTLSVELSGTAGLAPPTTPPHKANQDNPFRASPKLKSSCKTVVPPPSKKPRYSESSGTQGNNSTKKGPEQSELYAQLSKSSVLTGGHEERKTKRPSLRLFGDHDYCQSINSKTEILINISQELQDSRQLENKDVSSDWQGQICSSTDSDQCYLRETLEASKQVSPCSTRKQLQDQEIRAELNKHFGHPSQAVFDDEADKTGELRDSDFSNEQFSKLPMFINSGLAMDGLFDDSEDESDKLSYPWDGTQSYSLFNVSPSCSSFNSPCRDSVSPPKSLFSQRPQRMRARARAFARHRACARAPYSRSRSRSPGSRSSSRACYYYESSHYRHRTHRNAPLYVRSRARSPYARRPRYDSYEEYQHERLKREEYRREYEKRESERAKQRERQRQKAIEERRVIYVGKIRPDTTRTELRDRFEVFGEIEECTVNLRDDGDSYGFITYRYTCDAFAALENGYTLRRSNETDFELYFCGRKQFFKSNYADLDSNSDDFDPASTKSKYDSLDFDSLLKEAQRSLRR

SEQ ID NO: 6 - protein, 271 aa, Artificial Sequence
  Synthetic Construct
MAWDMCNQDSESVWSDIECAALVGEDQPACPDAPEADASELDVNDLDTDSFLGGLKWCSDQSEIISNQYNNEPSNIFEAIDEENEANLLAVLTETLDSLPVDEDGLPSFDALTDGDVTTDNEASPSSMPDGTPPPQEAEEPSLLAKLLLAPANTQLSYNECSGLSTQNHANHNHRIRTNPAIVATENSWSNKAKSICQQQKPQRRPCSELLKYLTTNDDPPHTKPTENRNSSRDKCTSKKKSHTQSQSQHLQAAPTTLSLPLTPESPNLFL

SEQ ID NO: 7 - protein, 271 aa, Artificial Sequence
  Synthetic Construct
MAWDMCNQDSESVWSDIECAALVGEDQPACPDAPEADASELDVNDLDTDSFLGGLKWCSDQSEIISNQYNNEPSNIFEAIDEENEANLLAVLTETADSAPADEDGLPSFDALTDGDVTTDNEASPSSMPDGTPPPQEAEEPSLLAKLLLAPANTQLSYNECSGLSTQNHANHNHRIRTNPAIVATENSWSNKAKSICQQQKPQRRPCSELLKYLTTNDDPPHTKPTENRNSSRDKCTSKKKSHTQSQSQHLQAAPTTLSLPLTPESPNLFL

SEQ ID NO: 8 - protein, 271 aa, Artificial Sequence
  Synthetic Construct
MAWDMCNQDSESVWSDIECAALVGEDQPACPDAPEADASELDVNDLDTDSFLGGLKWCSDQSEIISNQYNNEPSNIFEAIDEENEANLLAVLTETADSAPADEDGLPSFDALTDGDVTTDNEASPSSMPDGTPPPQEAEEPSLLAKLLLAPANTQLSYNECSGLSTQNHANHNHRIRTNPAIVATENSWSNKAKDICQQQKPQRRPCSELLKYLTTNDDPPHTKPTENRNSSRDKCTSKKKDHTQSQSQHLQAAPTDLSLPLDPEDPNDFL

SEQ ID NO: 9 - protein, 271 aa, Artificial Sequence
  Synthetic Construct
MAWDMCNQDSESVWSDIECAALVGEDQPACPDAPEADASELDVNDLDTDSFLGGLKWCSDQSEIISNQYNNEPSNIFEKIDEENEANLLAVLTETADSAPADEDGLPSFDALTDGDVTTDNEASPSSMPDGTPPPQEAEEPSLLKKLLLAPANTQLSYNECSGLSTQNHANHNHRIRTNPAIVKTENSWSNKAKDICQQQKPQRRPCSELLKYLTTNDDPPHTKPTENRNSSRDKCTSKKKDHTQSQSQHLQAKPTDLSLPLTPESPNLFL

SEQ ID NO: 10 - protein, 271 aa, Artificial Sequence
  Synthetic Construct
MAWDMCNQDSESVWSDIECAALVGEDQPACPDAPEADASELDVNDLDTDSFLGGLKWCSDQSEIISNQYNNEPSNIFEKIDEENEANLLAVLTETADSAPADEDGLPSFDALTDGDVTTDNEASPSSMPDGTPPPQEAEEPSLLKKLLLAPANTQLSYNECSGLSTQNHANHNHRIRTNPAIVKTENSWSNKAKSICQQQKPQRRPCSELLKYLTTNDDPPHTKPTENRNSSRDKCTSKKKSHTQSQSQHLQAKPTTLSLPLDPEDPNDFL

SEQ ID NO: 11 - protein, 271 aa, Artificial Sequence
  Synthetic Construct
MAWDMCNQDSESVWSDIECAALVGEDQPACPDAPEADASELDVNDLDTDSFLGGLKWCSDQSEIISNQYNNEPSNIFEKIDEENEANLLAVLTETLDSLPVDEDGLPSFDALTDGDVTTDNEASPSSMPDGTPPPQEAEEPSLLKKLLLAPANTQLSYNECSGLSTQNHANHNHRIRTNPAIVKTENSWSNKAKDICQQQKPQRRPCSELLKYLTTNDDPPHTKPTENRNSSRDKCTSKKKDHTQSQSQHLQAKPTDLSLPLTPESPNLFL

SEQ ID NO: 12 - protein, 271 aa, Artificial Sequence
  Synthetic Construct
MAWDMCNQDSESVWSDIECAALVGEDQPACPDAPEADASELDVNDLDTDSFLGGLKWCSDQSEIISNQYNNEPSNIFEKIDEENEANLLAVLTETLDSLPVDEDGLPSFDALTDGDVTTDNEASPSSMPDGTPPPQEAEEPSLLKKLLLAPANTQLSYNECSGLSTQNHANHNHRIRTNPAIVKTENSWSNKAKSICQQQKPQRRPCSELLKYLTTNDDPPHTKPTENRNSSRDKCTSKKKSHTQSQSQHLQAKPTTLSLPLDPEDPNDFL

SEQ ID NO: 13 - protein, 271 aa, Artificial Sequence
  Synthetic Construct
MAWDMCNQDSESVWSDIECAALVGEDQPLCPDLPELDLSELDVNDLDTDSFLGGLKWCSDQSEIISNQYNNEPSNIFEKIDEENEANLLAVLTETLDSLPVDEDGLPSFDALTDGDVTTDNEASPSSMPDGTPPPQEAEEPSLLKKLLLAPANTQLSYNECSGLSTQNHANHNHRIRTNPAIVKTENSWSNKAKDICQQQKPQRRPCSELLKYLTTNDDPPHTKPTENRNSSRDKCTSKKKDHTQSQSQHLQAKPTDLSLPLTPESPNLFL

SEQ ID NO: 14 - protein, 271 aa, Artificial Sequence
  Synthetic Construct
MAWDMCNQDSESVWSDIECAALVGEDQPLCPDLPELDLSELDVNDLDTDSFLGGLKWCSDQSEIISNQYNNEPSNIFEKIDEENEANLLAVLTETLDSLPVDEDGLPSFDALTDGDVTTDNEASPSSMPDGTPPPQEAEEPSLLKKLLLAPANTQLSYNECSGLSTQNHANHNHRIRTNPAIVKTENSWSNKAKSICQQQKPQRRPCSELLKYLTTNDDPPHTKPTENRNSSRDKCTSKKKSHTQSQSQHLQAKPTTLSLPLDPEDPNDFL

SEQ ID NO: 15 - protein, 271 aa, Artificial Sequence
  Synthetic Construct
MAWDMCNQDSESVWSDIECAALVGEDQPLCPDLPELDLSELDVNDLDTDSFLGGLKWCSDQSEIISNQYNNEPSNIFEAIDEENEANLLAVLTETLDSLPVDEDGLPSFDALTDGDVTTDNEASPSSMPDGTPPPQEAEEPSLLAKLLLAPANTQLSYNECSGLSTQNHANHNHRIRTNPAIVATENSWSNKAKSICQQQKPQRRPCSELLKYLTTNDDPPHTKPTENRNSSRDKCTSKKKSHTQSQSQHLQAAPTTLSLPLTPESPNLFL